(19) United States
(12) Patent Application Publication
(10) Pub. No.: US 2024/0183812 A1
(43) Pub. Date: Jun. 6, 2024

(54) POROUS MOISTURE-SENSITIVE MEMBER, HUMIDITY SENSOR, AND RESPIRATION SENSING SYSTEM

(71) Applicant: Murata Manufacturing Co., Ltd., Nagaokakyo-shi (JP)

(72) Inventors: Hiroshi SHIRAKI, Nagaokakyo-shi (JP); Yuki NISHIKORI, Nagaokakyo-shi (JP)

(21) Appl. No.: 18/419,888

(22) Filed: Jan. 23, 2024

Related U.S. Application Data
(63) Continuation of application No. PCT/JP2023/009263, filed on Mar. 10, 2023.

(30) Foreign Application Priority Data
Mar. 31, 2022 (JP) 2022-060776

(51) Int. Cl. G01N 27/12; A61B 5/08; G01N 27/22

(52) U.S. Cl. CPC G01N 27/125; A61B 5/0816; G01N 27/121; G01N 27/227; A61B 2562/029

(57) ABSTRACT

A porous moisture-sensitive member for a humidity sensor, the porous moisture-sensitive member including: a resin base material; and an inorganic filler, wherein the porous moisture-sensitive member contains surface pores and internal pores, and an average pore size of the surface pores is 0.1 μm or more, and a humidity sensor and a respiration sensing system using the same.

### CROSS REFERENCE TO RELATED APPLICATIONS

The present application is a continuation of International application No. PCT/JP2023/009263, filed Mar. 10, 2023, which claims priority to Japanese Patent Application No. 2022-060776, filed Mar. 31, 2022, the entire contents of each of which are incorporated herein by reference.

### TECHNICAL FIELD

The present disclosure relates to a porous moisture-sensitive member, and a humidity sensor and a respiration sensing system including the porous moisture-sensitive member.

### BACKGROUND ART

Humidity sensors are being used in a wide range of applications such as printers, air conditioners, air cleaners, microwave ovens, and automotive applications. The recent advances on the IoT technology have increased the need to monitor not only physical information such as location and speed, but also chemical information such as temperature, humidity, and gas.

Patent Document 1 discloses that a humidity sensor in which a moisture-sensitive part composed of potassium fluoride is provided on a surface of a polytetrafluoroethylene porous body has advantages of being superior in heat resistance, resistant to adhesion of dirt, simple in maintenance, and low in electric resistance.

Patent Document 2 describes an example of respiration sensing using a millimeter-wave sensor, and discloses that respiration can be detected by analyzing a vibration component due to respiration when a millimeter wave is applied to a human abdomen from a sensing unit attached to a wash basin.

Patent Document 3 describes a method of sensing sleep apnea syndrome using a humidity sensor, and discloses that a humidity sensor is installed near the oral cavity of a subject (for example, around the mouth, between the mouth and the nose) and a humidity change caused by respiration is measured, whereby a respiratory state during sleep can be sensed.
Patent Document 1: Japanese Patent Application Laid-Open No. H1-129151
Patent Document 2: Japanese Patent Application Laid-Open No. 2009-22489
Patent Document 3: Japanese Patent Application Laid-Open No. 2005-270570

### SUMMARY OF THE INVENTION

The humidity sensor in Patent Document 1 is one in which porous polytetrafluoroethylene whose prescribed surface is surface-treated with potassium fluoride is used, but neither its internal structure nor porosity has been studied at all. In recent years, along with the evolution of IoT technology, applications of humidity sensors are expanding, and further improvement in performance of humidity sensors is required, and in particular, further improvement in such performance as sensitivity and responsiveness is required. Conventionally, a polymer material is commonly used as a moisture-sensitive member in a humidity sensor, and the influence of the addition of an inorganic filler in a moisture-sensitive member on sensor characteristics and the influence of the porosity of a moisture-sensitive member have not been sufficiently studied so far.

Since the respiration sensing using a millimeter-wave sensor in Patent Document 2 uses a minute vibration component of the abdomen due to respiration, when the subject moves or vibrates for some reason, these vibrations become noise components and respiration cannot be accurately measured. Furthermore, in Patent Document 2, even if the respiration rate can be measured, it is difficult to measure the form of respiration.

In the method of sensing respiration while installing a humidity sensor near the oral cavity of a subject (for example, around the mouth, between the mouth and the nose) in Patent Document 3, it is necessary to install the sensor around the oral cavity, and there is a problem that a member for fixing the sensor near the oral cavity is required for sensing respiration. In addition, it is also troublesome to dispose the sensor and the fixing member near the oral cavity. In addition, it is difficult to measure the respiration rate using a conventional humidity sensor because the response speed of the sensor is slow.

An object of the present disclosure is to provide a novel moisture-sensitive member to be used for a humidity sensor that exhibits good sensitivity and responsiveness, and a humidity sensor and a respiration sensing system using the same.

Aspects of the present disclosure are as follows.

(1) A porous moisture-sensitive member for a humidity sensor, the porous moisture-sensitive member comprising: a resin base material; and an inorganic filler, wherein the porous moisture-sensitive member includes surface pores and internal pores, and the surface pores have an average pore size of 0.1 μm or more.

(2) A humidity sensor including: a first electrode; a second electrode; and the porous moisture-sensitive member of (1) between the first electrode and the second electrode.

(3) A respiration sensing system for sensing respiration of a subject, the respiration sensing system comprising: the humidity sensor of (2) disposed within a sphere region defined by a sphere that has a center as an exhalation source of the subject and has a radius of 150 cm or less.

The humidity sensor including the porous moisture-sensitive member in the present disclosure exhibits good sensitivity and responsiveness.

The humidity sensor in the present disclosure exhibits good sensitivity and responsiveness, and can be suitably used for respiration sensing.

### BRIEF EXPLANATION OF THE DRAWINGS

FIG. 1 shows the correlation between the amount of Ni filler contained in a moisture-sensitive film (porous moisture-sensitive member) and the sensitivity (upper diagram) or responsiveness (lower diagram) of a humidity sensor in one embodiment of the present disclosure.

FIG. 2 shows surface SEM images of moisture-sensitive films in one embodiment of the present disclosure.

FIG. 3 shows a correlation between the amount of tetraglyme used for a moisture-sensitive film and the sensitivity of a humidity sensor in one embodiment of the present disclosure.

FIG. 4 shows a correlation between the amount of a surfactant used at the time of forming a moisture-sensitive film and the sensitivity of a humidity sensor in one embodiment of the present disclosure.

FIG. 5 shows a correlation between the average pore size of surface pores of a moisture-sensitive film and sensitivity of a humidity sensor in one embodiment of the present disclosure.

FIG. 6 shows sectional SEM images of moisture-sensitive films in embodiments of the present disclosure.

FIG. 7 shows a correlation between the amount of CCTO filler contained in a moisture-sensitive film and the relative permittivity of a porous moisture-sensitive member according to one embodiment of the present disclosure.

FIG. 8 shows a correlation between the amount of CCTO filler contained in a moisture-sensitive film and the sensitivity of a humidity sensor in one embodiment of the present disclosure.

FIG. 9A shows measurement data in a respiration sensing system in one embodiment of the present disclosure.

FIG. 9B shows a Fourier transform result of measurement data in the respiration sensing system in one embodiment of the present disclosure.

FIG. 10 shows measurement data and a Fourier transform result in a respiration sensing system in one embodiment of the present disclosure.

FIG. 11 illustrates a schematic diagram of a region (circular cone A region) within which a humidity sensor is disposed in a respiration sensing system in one embodiment of the present disclosure.

FIG. 12 shows measurement data and a Fourier transform result in a respiration sensing system in one embodiment of the present disclosure.

FIG. 13 shows measurement data and a Fourier transform result in a respiration sensing system in one embodiment of the present disclosure.

FIG. 14 shows measurement data and a Fourier transform result in a respiration sensing system in one embodiment of the present disclosure.

FIG. 15 illustrates a schematic diagram of a region (circular cone B region) in which a humidity sensor is disposed in a respiration sensing system in one embodiment of the present disclosure.

FIG. 16 shows measurement data and a Fourier transform result in a respiration sensing system in one embodiment of the present disclosure.

FIG. 17 illustrates a schematic diagram of a region (circular cone C region) in which a humidity sensor is disposed in a respiration sensing system in one embodiment of the present disclosure.

FIG. 18 shows measurement data and a Fourier transform result in a respiration sensing system in one embodiment of the present disclosure.

FIG. 19 shows measurement data and a Fourier transform result in a respiration sensing system in one embodiment of the present disclosure.

FIG. 20 shows measurement data and a Fourier transform result in a respiration sensing system in one embodiment of the present disclosure.

FIG. 21A shows measurement data in a respiration sensing system in one embodiment of the present disclosure.

FIG. 21B shows measurement data in a respiration sensing system in one embodiment of the present disclosure.

FIG. 21C shows measurement data in a respiration sensing system in one embodiment of the present disclosure.

FIG. 22 shows correlations between temperature, humidity, and time when a wind from an air conditioner directly hits and when a wind from an air conditioner does not directly hit in one embodiment of the present disclosure.

FIG. 23 shows measurement data in a respiration sensing system in one embodiment of the present disclosure.

### DETAILED DESCRIPTION OF THE PREFERRED EMBODIMENTS

Hereinafter, a porous moisture-sensitive member, a humidity sensor, and so on according to embodiments of the present disclosure will be described in more detail with reference to drawings, as necessary. However, unnecessarily detailed description may be omitted. For example, a detailed description on already well-known matters or a repeated description of substantially the same configuration may be omitted. This is to avoid unnecessary redundancy of the description and to facilitate understanding by those skilled in the art.

The applicant provides the accompanying drawings and the following description in order for those skilled in the art to fully understand the present disclosure, and does not intend to limit the subject matter described in the claims. Various elements in the drawings are merely shown schematically and exemplarily for the understanding of the present disclosure, and appearance, dimensional ratios, and so on may be different from actual ones.

In the present description, a porous moisture-sensitive member may be simply referred to as a moisture-sensitive member, and a humidity sensor may be simply referred to as a humidity sensor.

### <Porous Moisture-Sensitive Member>

The porous moisture-sensitive member in the present disclosure is suitably used for a humidity sensor. In the present description, a “member” refers to an object constituting a humidity sensor, and can be reworded as “material”, “component”, “part”, “configuration”, or the like.

The porous moisture-sensitive member in the present disclosure comprises a resin base material and an inorganic filler. The porous moisture-sensitive member is a so-called composite material, and has a porous structure in which an inorganic filler is dispersed and composited in a porous resin base material network. As a result of using the porous moisture-sensitive member in the present disclosure as a moisture-sensitive member of a humidity sensor, coming in and out of water molecules with the moisture-sensitive member easily occurs, so that high-speed responsiveness (high-speed responsiveness and/or high-speed recovery) can be achieved. In addition, it is considered that as a result of using the porous moisture-sensitive member in the present disclosure, the amount of adsorption of water molecules is increased, and the sensor sensitivity is also improved.

[Shape, etc. of porous moisture-sensitive member]

The shape of the porous moisture-sensitive member is usually a film shape or a sheet shape while depending on the structure of the humidity sensor to be used. The thickness of the porous moisture-sensitive member may be 0.5 μm or more, 1 μm or more, 2.5 μm or more, 5 μm or more, or 10 μm or more, and is preferably 1 μm or more. The thickness of the porous moisture-sensitive member may be 1000 μm or less, 500 μm or less, 100 μm or less, 50 μm or less, 10 μm or less, or 5 μm or less, and is preferably 50 μm or less. Falling in the aforementioned range is suitable from the viewpoint of sensitivity or responsiveness of a humidity sensor.

### [Porosity]

The porous moisture-sensitive member in the present disclosure contains a large number of pores (voids). It is preferable that pores are distributed throughout the porous moisture-sensitive member, and it is preferable that the porous moisture-sensitive member contain not only surface pores but also internal pores. The “surface pore” refers to a pore that is open to the outside of the porous moisture-sensitive member on a surface of the porous moisture-sensitive member. The “internal pore” refers to a pore that is present inside the porous moisture-sensitive member and is not open to the outside of the porous moisture-sensitive member. The internal pore may be flat in the horizontal direction. The internal pore may be interconnected with an adjacent pore (a pore directly or indirectly communicating with a surface pore also is an internal pore as long as the pore is not directly open to the outside of the porous moisture-sensitive member). Owing to that a moisture-sensitive member is porous, a large number of water molecules are likely to be adsorbed to the moisture-sensitive member, so that a change in physical properties of the moisture-sensitive member due to a humidity change is increased and sensitivity can be improved. Owing to containing the surface pore and the internal pores, the sensitivity or responsiveness of the humidity sensor can be improved.

### (Average Pore Size of Surface Pores)

The average pore size of the surface pores may be 0.1 μm or more, 0.5 μm or more, 1 μm or more, 3 μm or more, or 5 μm or more, and is preferably 1 μm or more. The average pore size of the surface pores may be 50 μm or less, 25 μm or less, 10 μm or less, 5 μm or less, 2.5 μm or less, 1.2 μm or less, 0.6 μm or less, or 0.2 μm or less. The average pore size of the surface pores may be ½ or less, ⅓ or less, ¼ or less, or ⅕ or less of the thickness of the porous moisture-sensitive member from the viewpoint of adhesion to a substrate. Falling in the aforementioned range is suitable from the viewpoint of sensitivity or responsiveness of a humidity sensor. When the pore size is large, water molecules easily come in and out, so that responsiveness is improved, and water molecules can be adsorbed in multiple layers on a pore surface, so that sensitivity can be improved. The average pore size of the surface pores can be determined by microscopic observation. A surface of the porous moisture-sensitive member is observed with a microscope, and the longest size of each surface pore observed can be regarded as the pore size of each surface pore. The pore sizes of all the surface pores existing in a visual field are measured, then the operation of moving the visual field and measuring pore sizes again is repeated, and the average value for 100 or more surface pores is taken as the average pore size of the surface pores.

### (Density of Surface Pores)

The density of surface pores having a pore size of 0.1 μm or more (the pore size may be ½ or less, ⅓ or less, ¼ or less, or ⅕ or less of the thickness of the porous moisture-sensitive member, or 10 μm or less, 5 μm or less, 2.5 μm or less, or 1 μm or less, and is particularly 10 μm or less or 5 μm or less) may be 1 pore/100 μm² or more, 5 pores/100 μm² or more, 10 pores/100 μm² or more, 50 pores/100 μm² or more, 100 pores/100 μm² or more, 250 pores/100 μm² or more, 500 pores/100 μm² or more, or 750 pores/100 μm² or more, and is preferably 50 pores/100 μm² or more, more preferably 250 pores/100 μm² or more. The density of surface pores having a pore size of 0.1 μm or more (the pore size may be ½ or less, ⅓ or less, ¼ or less, or ⅕ or less of the thickness of the porous moisture-sensitive member, or 10 μm or less, 5 μm or less, 2.5 μm or less, or 1 μm or less, and is particularly 10 μm or less or 5 μm or less) may be 2500 pores/100 μm² or less, 1000 pores/100 μm² or less, 500 pores/100 μm² or less, 250 pores/100 μm² or less, or 100 pores/100 μm² or less. Falling in the aforementioned range is suitable from the viewpoint of sensitivity or responsiveness of a humidity sensor. When the pore size is large, water molecules easily come in and out, so that responsiveness is improved, and water molecules can be adsorbed in multiple layers on a pore surface, so that sensitivity can be improved. The density of surface pores can be determined by measuring the density of surface pores from a microscopic image of a surface of the porous moisture-sensitive member having at least a visual field of 100 μm² or more.

### (Pore Section)

It is preferable that a pore section exists in any section of the porous moisture-sensitive member. The section may be a thickness direction section (vertical direction section). The sectional size of an existing pore section may be 0.1 μm or more, 0.5 μm or more, 1 μm or more, 2 μm or more, 3 μm or more, 4 μm or more, or 5 μm or more, and is preferably 1 μm or more. The size of the existing pore section may be ⅔ or less, or ½ or less of the thickness of the porous moisture-sensitive member, or 50 μm or less, 25 μm or less, 10 μm or less, 7.5 μm or less, or 5 μm or less, and is preferably 10 μm or less. Falling in the aforementioned range is suitable from the viewpoint of sensitivity or responsiveness of a humidity sensor. When the pore size is large, water molecules easily come in and out, so that responsiveness is improved, and water molecules can be adsorbed in multiple layers on a pore surface, so that sensitivity can be improved. The sectional size of a pore section in a section of the porous moisture-sensitive member can be determined by microscopic observation. Pore sections are observed with a microscope, and the longest size of each pore section observed can be regarded as the sectional size of each pore section. Examples of the method for section observation include a method in which a porous moisture-sensitive member is vertically embedded in an epoxy resin, the embedded surface is mechanically polished, and then observed with an SEM (scanning electron microscope), and a method in which a sample for observation is cut out using an FIB (focused ion beam) apparatus so that a section of the porous moisture-sensitive member can be seen, and the section is observed with an SEM (scanning electron microscope).

### (Density of Pore Sections)

In an arbitrary section of the porous moisture-sensitive member, the density of pore sections having a sectional size of 1 μm or more (the sectional size may be ⅔ or less or ½ or less of the thickness of the multi-hole moisture-texture-like member, or 10 μm or less, 5 μm or less, 2.5 μm or less, or 1 μm or less, and is particularly 10 μm or less) may be 10 pore sections/100 μm² or more, 20 pore sections/100 μm² or more, 30 pore sections/100 μm² or more, 40 pore sections/100 μm² or more, 50 pore sections/100 μm² or more, 60 pore sections/100 μm² or more, or 70 pore sections/100 μm² or more, and is preferably 50 pore sections/100 μm². In the section of the porous moisture-sensitive member, the density of pore sections having a sectional size of 1 μm or more (the sectional size may be ⅔ or less or ½ or less of the thickness of the multi-hole moisture-texture-like member, or 10 μm or less, 5 μm or less, 2.5 μm or less, or 1 μm or less, and is particularly 10 μm or less) may be 100 pore sections/100 μm² or less, 90 pore sections/100 μm² or less, 80 pore sections/100 μm² or less, or 70 pore sections/100 μm² or less. The section may be a thickness direction section (vertical direction section). Falling in the aforementioned range is suitable from the viewpoint of sensitivity or responsiveness of a humidity sensor. When the pore size is large, water molecules easily come in and out, so that responsiveness is improved, and water molecules can be adsorbed in multiple layers on a pore surface, so that sensitivity can be improved. The density of pore sections can be determined by measuring the density of pore sections from a section of the porous moisture-sensitive member having at least a visual field of 100 μm² or more.

### (Specific Surface Area)

The specific surface area of the porous moisture-sensitive member may be 0.1 m²/g or more, 0.5 m²/g or more, 1 m²/g or more, 2 m²/g or more, 3 m²/g or more, 5 m²/g or more, or 7.5 m²/g or more. The specific surface area of the porous moisture-sensitive member may be 50 m²/g or less, 20 m²/g or less, 10 m²/g or less, 5 m²/g or less, 2 m²/g or less, or 1.5 m²/g or less, and is preferably 2 m²/g or less. Falling in the aforementioned range is suitable from the viewpoint of sensitivity or responsiveness of a humidity sensor. The specific surface area can be determined by the BET method.

### (Porosity)

The porosity of the porous moisture-sensitive member may be 5% by volume or more, 10% by volume or more, 20% by volume or more, 30% by volume or more, 40% by volume or more, or 50% by volume or more, 60% by volume or more, 70% by volume or more, 80% by volume or more, or 90% by volume or more, is preferably 10% by volume or more, or 30% by volume or more, and is more preferably 60% by volume or more. The porosity of the porous moisture-sensitive member may be 99% by volume or less, 90% by volume or less, 80% by volume or less, 70% by volume or less, 60% by volume or less, 50% by volume or less, 40% by volume or less, or 30% by volume or less, and is preferably 90% by volume or less. Falling in the aforementioned range is suitable from the viewpoint of sensitivity or responsiveness of a humidity sensor. When the porosity is large, water molecules easily come in and out, so that the responsiveness of the humidity sensor is improved, and water molecules can be adsorbed in multiple layers on a pore surface, so that the sensitivity can be improved. The porosity is defined by the ratio of the volume of pores to the entire volume of the porous moisture-sensitive member, and the porosity can be determined by an optical method, a water evaporation method, SEM image observation, an Archimedes method, or the like.

### [Relative Permittivity]

The relative permittivity of the porous moisture-sensitive member may be 1.2 or more, 1.4 or more, 1.6 or more, 1.8 or more, 2.0 or more, 2.2 or more, 2.4 or more, 2.7 or more, 3.0 or more, or 3.3 or more, and a higher relative permittivity is more preferable. The relative permittivity of the porous moisture-sensitive member may be 100 or less, 80 or less, 60 or less, 40 or less, 20 or less, 10 or less, 7.5 or less, or 5.0 or less. Falling in the aforementioned range is suitable from the viewpoint of sensitivity or responsiveness of a humidity sensor. It is considered that as the relative permittivity of the porous moisture-sensitive member increases, more water molecules can be adsorbed, and as the physical property change of the moisture-sensitive member due to a humidity change increases, sensitivity can be improved.

### [Resin Base Material]

The resin base material may be a thermoplastic resin, a thermosetting resin, or a combination thereof. The resin base material may be a homopolymer, a copolymer such as a star-shaped block copolymer, a graft copolymer, an alternating block copolymer, or a random copolymer, an ionomer, a dendrimer, or the like. Examples of the resin base material include polyamide, polyimide, polyamideimide, polyurethane, polyether, polyetherimide, polyether ketone, polycarbonate, polyester, polyacryl, polyolefin, polyvinyl alcohol, polyvinyl halide, polysiloxane, and modified cellulose, and specific examples include aromatic polyimide, aromatic polyamideimide, aromatic polyamide, aromatic polyether, polyethylene terephthalate, cellulose acetate butyrate (CAB), polymethyl methacrylate (PMMA), and vinyl crotonate. These may be used singly, or two or more of them may be used in combination.

The resin base material may have high hygroscopicity from the viewpoint of enhancing the interaction with water molecules of the porous moisture-sensitive member and improving the sensitivity and responsiveness of the humidity sensor. For example, the resin base material may be a resin containing a highly polar group such as an amide group, an imide group, an amino group, a carboxy group, a hydroxy group, a phosphoric acid group, a sulfonic acid group, or a nitrile group in a repeating unit, particularly a group capable of forming a hydrogen bond, particularly a resin containing an amide group or an imide group.

### (Amount of Resin Base Material)

The amount of the resin base material may be 10% by weight or more, 20% by weight or more, 30% by weight or more, 40% by weight or more, 50% by weight or more, 60% by weight or more, or 70% by weight or more with respect to the porous moisture-sensitive member, and is preferably 50% by weight or more. The amount of the resin base material may be 99% by weight or less, 90% by weight or less, 80% by weight or less, 70% by weight or less, 60% by weight or less, 50% by weight or less, 40% by weight or less, or 30% by weight or less with respect to the porous moisture-sensitive member.

The amount of the resin base material may be 5% by volume or more, 10% by volume or more, 15% by volume or more, 20% by volume or more, 25% by volume or more, 30% by volume or more, or 50% by volume or more with respect to the porous moisture-sensitive member, and is preferably 20% by volume or more. The amount of the resin base material may be 75% by volume or less, 60% by volume or less, 50% by volume or less, 40% by volume or less, 30% by volume or less, 20% by volume or less, or 10% by volume or less with respect to the porous moisture-sensitive member.

### [Inorganic Filler]

The inorganic filler may be in the form of particles (spherical, flaky, plate-like, etc.). The average particle size of the inorganic filler may be 50 nm or more, 100 nm or more, 300 nm or more, 500 nm or more, or 1000 nm or more. The average particle size of the inorganic filler may be 10,000 nm or less, 5000 nm or less, 3000 nm or less, 1000 nm or less, or 500 nm or less. The average particle size of the inorganic filler can be determined by microscopic observation. The inorganic filler is observed with a microscope, and the longest size of the observed inorganic filler can be regarded as the particle size of each inorganic filler. The particle sizes of all the inorganic fillers existing in a visual field are measured, then the operation of moving the visual field and measuring particle sizes again is repeated, and the average value thereof is taken as the average particle size of the inorganic filler particles.

Examples of the inorganic filler include a ceramic-based filler and a metal-based filler. Examples of the ceramic-based filler include oxide-based fillers such as silica, alumina, mica, talc, titania, titanates (potassium titanate, barium titanate, bismuth titanate, magnesium titanate, and the like), zirconia, zirconates, zinc oxide, iron oxide, and ferrite; hydroxide-based fillers such as aluminum hydroxide and hydroxyapatite; carbide-based fillers such as silicon carbide, aluminum carbide, and calcium carbide; nitride-based fillers such as silicon nitride and aluminum nitride; carbonate-based fillers such as calcium carbonate and magnesium carbonate; sulfate-based fillers such as barium sulfate and aluminum sulfate; phosphate-based fillers such as calcium phosphate and aluminum phosphate; halide-based fillers such as aluminum fluoride, carbon fluoride, and fluorite; and carbon-based fillers such as carbon black, graphite, graphene, and carbon nanotubes. Examples of the metal-based filler include aluminum, silver, copper, iron, nickel, zinc, stainless steel, brass, and alloys containing these metals. These may be used singly, or two or more of them may be used in combination.

The inorganic filler may be a material having a dipole moment such as a ferroelectric or a material having oxygen deficiency. The inorganic filler may comprise a high-dielectric material. The relative permittivity of the high-dielectric material at 25° C. and 1 kHz may be 5 or more, 10 or more, 100 or more, 500 or more, or 1000 or more. Examples of the inorganic filler include barium titanate (BaTiO₃), strontium titanate (SrTiO₃), barium strontium titanate, strontium-doped lanthanum manganate, lanthanum aluminum oxide (LaAlO₃), lanthanum strontium copper oxide (LSCO), yttrium barium copper oxide (YBa₂Cu₃O₇), lead zirconium titanate, lanthanum-modified lead zirconium titanate, lead magnesium niobate-lead titanate, and calcium copper titanium oxide (CCTO). The inorganic filler may comprise a material having a perovskite structure. The perovskite structure is an ABO₃ type crystal structure that ideally has a cubic unit lattice and is composed of metal A disposed at each vertex of a cubic crystal, metal B disposed at the body center, and oxygen O disposed at each face center of the cubic crystal. The perovskite structure also includes tetragonal, orthorhombic, and rhombohedral crystals, which are distorted cubic crystals. The inorganic filler may be surface-modified with a surface treatment agent (for example, a silane coupling agent) or the like. The inorganic filler may be used singly, or two or more thereof may be used in combination.

### (Amount of Inorganic Filler)

The amount of the inorganic filler may be 1% by weight or more, 10% by weight or more, 20% by weight or more, 30% by weight or more, 40% by weight or more, 50% by weight or more, 60% by weight or more, 70% by weight or more, 80% by weight or more, or 90% by weight or more with respect to the porous moisture-sensitive member, and is preferably 30% by weight or more, more preferably 50% by weight or more, particularly 70% by weight or more from the viewpoint of the sensitivity or responsiveness of a humidity sensor. The amount of the inorganic filler may be 99% by weight or less, 90% by weight or less, 80% by weight or less, 70% by weight or less, 60% by weight or less, 50% by weight or less, 40% by weight or less, or 30% by weight or less with respect to the porous moisture-sensitive member.

The amount of the inorganic filler may be 1% by weight or more, 10% by weight or more, 20% by weight or more, 30% by weight or more, 40% by weight or more, 50% by weight or more, 60% by weight or more, 70% by weight or more, 80% by weight or more, or 90% by weight or more with respect to the resin base material, and is preferably 30% by weight or more, more preferably 50% by weight or more, particularly 70% by weight or more from the viewpoint of the sensitivity or responsiveness of a humidity sensor. The amount of the inorganic filler may be 99% by weight or less, 90% by weight or less, 80% by weight or less, 70% by weight or less, 60% by weight or less, 50% by weight or less, 40% by weight or less, or 30% by weight or less with respect to the resin base material.

The amount of the inorganic filler may be 1% by volume or more, 10% by volume or more, 20% by volume or more, 30% by volume or more, 40% by volume or more, or 50% by volume or more with respect to the resin base material, and is preferably 10% by volume or more, 30% by volume or more, or 50% by volume or more from the viewpoint of the sensitivity or responsiveness of a humidity sensor. The amount of the inorganic filler may be 99% by volume or less, 90% by volume or less, 80% by volume or less, 70% by volume or less, 60% by volume or less, 50% by volume or less, 40% by volume or less, or 30% by volume or less with respect to the resin base material.

### [Surfactant]

The porous moisture-sensitive member may comprise a surfactant. Owing to comprising the surfactant, the pore size and the pore density can be adjusted. Examples of the surfactant include anionic, cationic, amphoteric, and nonionic surfactants. The surfactant may comprise a fluorine-based surfactant. Owing to comprising the fluorine-based surfactant, water repellency, oil repellency, antifouling property, and the like are imparted, and good sensitivity and responsiveness of a humidity sensor can be provided. In addition, by using a hydrophobic ionic surfactant, pores having a larger size can be formed. The surfactant may be used singly, or two or more thereof may be used in combination.

### (Amount of Surfactant)

The amount of the surfactant may be 0.1% by weight or more, 0.5% by weight or more, 1% by weight or more, 2% by weight or more, 3% by weight or more, 4% by weight or more, or 5% by weight or more with respect to the porous moisture-sensitive member, and is preferably 1% by weight or more. The amount of the surfactant may be 25% by weight or less, 20% by weight or less, 15% by weight or less, 10% by weight or less, or 7.5% by weight or less with respect to the porous moisture-sensitive member.

The amount of the surfactant may be 0.1% by weight or more, 0.5% by weight or more, 1% by weight or more, 2% by weight or more, 3% by weight or more, 4% by weight or more, or 5% by weight or more with respect to the inorganic filler, and is preferably 1% by weight or more. The amount of surfactant may be 25% by weight or less, 20% by weight or less, 15% by weight or less, 10% by weight or less, or 7.5% by weight or less with respect to the inorganic filler.

### [Silane Coupling Agent]

The porous moisture-sensitive member in the present disclosure may contain a silane coupling agent or be treated with a silane coupling agent. When the porous moisture-sensitive member contains a silane coupling agent or is treated with a silane coupling agent, adhesiveness between the resin base material and the inorganic filler is improved, and good sensitivity and responsiveness of a humidity sensor can be provided.

The type of the silane coupling agent is not particularly limited, and examples thereof include vinyl-based silane coupling agents such as vinyltrimethoxysilane, vinyltriethoxysilane, vinyltris(2-methoxyethoxy)silane, allyltrichlorosilane, allyltrimethoxysilane, allyltriethoxysilane, diethoxymethylvinylsilane, trichlorovinylsilane, and triethoxyvinylsilane; epoxy-based silane coupling agents such as 2-(3,4-epoxycyclohexyl)ethyltrimethoxysilane, 3-glycidoxypropyltrimethoxysilane, and 3-glycidoxypropyltriethoxysilane; methacrylic silane coupling agents such as methacryloxymethyltrimethoxysilane, methacryloxymethyltriethoxysilane, methacryloxymethylmethyldimethoxysilane, methacryloxymethyldimethylmethoxysilane, γ-methacryloxypropyltrimethoxysilane, γ-methacryloxypropyltriethoxysilane, γ-methacryloxypropylmethyldimethoxysilane, γ-methacryloxypropylmethyldiethoxysilane, and γ-methacryloxypropyldimethylmethoxysilane; acrylic silane coupling agents such as acryloxymethyltrimethoxysilane, acryloxymethyltriethoxysilane, acryloxymethyldimethoxysilane, acryloxymethyldimethylmethoxysilane, γ-acryloxypropyltrimethoxysilane, γ-acryloxypropyltriethoxysilane, γ-acryloxypropylmethyldimethoxysilane, γ-acryloxypropylmethyldiethoxysilane, and γ-acryloxypropyldimethylmethoxysilane; amino-based silane coupling agents such as 3-aminopropyltriethoxysilane, 3-aminopropyltrimethoxysilane, N-(2-aminoethyl)-3-aminopropyltrimethoxysilane, N-(2-aminoethyl)-3-aminopropylmethyldimethoxysilane, and 3-(N-phenyl)aminopropyltrimethoxysilane; mercapto-based silane coupling agents such as 3-mercaptopropyltrimethoxysilane and 3-mercaptopropyltriethoxysilane; haloalkyl-based silane coupling agents such as 3-chloropropyltrimethoxysilane and 3-chloropropyltriethoxysilane; and sulfide-based silane coupling agents such as bis(3-triethoxysilylpropyl)disulfide (abbreviation: TESPD) and bis(3-triethoxysilylpropyl)tetrasulfide.

### (Amount of Silane Coupling Agent)

The amount of the silane coupling agent may be 0.1% by weight or more, 0.5% by weight or more, 1% by weight or more, 2% by weight or more, 3% by weight or more, 4% by weight or more, or 5% by weight or more with respect to the porous moisture-sensitive member, and is preferably 1% by weight or more. The amount of the silane coupling agent may be 25% by weight or less, 20% by weight or less, 15% by weight or less, 10% by weight or less, or 7.5% by weight or less with respect to the porous moisture-sensitive member.

The amount of the silane coupling agent may be 0.1% by weight or more, 0.5% by weight or more, 1% by weight or more, 2% by weight or more, 3% by weight or more, 4% by weight or more, or 5% by weight or more with respect to the inorganic filler, and is preferably 1% by weight or more. The amount of the silane coupling agent may be 25% by weight or less, 20% by weight or less, 15% by weight or less, 10% by weight or less, or 7.5% by weight or less with respect to the inorganic filler.

### [Other Components]

The porous moisture-sensitive member may contain other components. Examples of other components include a plasticizer, a lubricant, a colorant (pigment, dye, etc.), an ultraviolet absorber, an antioxidant, an antiaging agent, a foaming agent, a defoaming agent, a reinforcing agent, a flame retardant, an antistatic agent, a surfactant, a surface treatment agent, a water repellent agent, an oil repellent agent, and an antifouling agent. These may be used singly, or two or more of them may be used in combination.

### (Amount of Other Components)

The amount of other components can be appropriately selected as long as the effects of the present disclosure are not impaired. The amount of each other component may be 0.1% by weight or more, 0.3% by weight or more, 0.5% by weight or more, 0.7% by weight or more, or 1% by weight or more with respect to the porous moisture-sensitive member. The amount of the other component may be 10% by weight or less, 7.5% by weight or less, 5% by weight or less, 3% by weight or less, 2% by weight or less, or 1% by weight or less with respect to the porous moisture-sensitive member.

[Method for Producing Porous Moisture-Sensitive member]

The method for producing the porous moisture-sensitive member may include a dispersion preparation step and an application and drying step. In the step of preparing a dispersion, a resin base material, an inorganic filler, and other components as necessary are dispersed in a solvent to afford a dispersion. In the application and drying step, a porous moisture-sensitive member can be obtained by applying the obtained dispersion to a substrate (for example, a base plate and an electrode) and further drying the dispersion. When more active foaming is intended, a foaming agent may be used as a component of the dispersion.

As the solvent of the dispersion, a solvent capable of dissolving a resin can be used, but it is preferable that the solvent includes an ether-based solvent, especially a glycol ether-based solvent, from the viewpoint of obtaining a porous moisture-sensitive member having a favorable pore density. The amount of the ether-based solvent may be 10% by weight or more, 30% by weight or more, 50% by weight or more, or 60% by weight or more of the solvent, and is preferably 30% by weight or more. The amount of the ether-based solvent may be 70% by weight or less, 60% by weight or less, 50% by weight or less, or 40% by weight or less, 30% by weight or less, or 20% by weight or less of the solvent.

### <Humidity Sensor>

The present disclosure further provides a humidity sensor. The humidity sensor in the present disclosure may include: a first electrode; a second electrode; and a porous moisture-sensitive member provided between the first electrode and the second electrode.

The humidity sensor functions as a humidity sensor on the principle that the porous moisture-sensitive member adsorbs moisture to change the physical properties, so that the electrical characteristics between the first electrode and the second electrode change depending on the amount of moisture contained in the ambient atmosphere (that is, humidity). In particular, since the porous moisture-sensitive member in the present disclosure is superior in water adsorption/desorption property, the humidity sensor in the present disclosure is superior in sensitivity and responsiveness.

The humidity sensor in the present disclosure may be of a resistance change type or a capacitance change type. A resistance change type humidity sensor measures a resistance change of a moisture-sensitive member due to a humidity change, and a capacitance change type humidity sensor measures a capacitance change of a moisture-sensitive member due to a humidity change. The humidity sensor in the present disclosure is particularly suitable as a capacitance change type humidity sensor from the viewpoint of performance such as sensitivity and responsiveness.

The humidity sensor may include a base plate, and the first electrode and the porous moisture-sensitive member may be in contact with each other as a result of forming the first electrode on the base plate. In this case, the porous moisture-sensitive member may include a base plate, a first electrode formed on the base plate, a porous moisture-sensitive member formed on the first electrode, and a second electrode formed on the porous moisture-sensitive member.

As another embodiment, the humidity sensor may include a base plate, a first electrode and a second electrode formed on the base plate, and a porous moisture-sensitive member provided between the first electrode and the second electrode. As still another embodiment, the humidity sensor may include a base plate, a porous moisture-sensitive member formed on the base plate, and a first electrode and a second electrode formed on the porous moisture-sensitive member.

One or both of the first electrode and the second electrode may be a comb-shaped electrode. The first electrode and the second electrode may be comb-shaped electrodes and disposed to engage with each other. For example, in the case of using comb-shaped electrodes, the humidity sensor may include a base plate, a first electrode and a second electrode formed on the base plate, and a porous member provided between the first electrode and the second electrode.

The first electrode may be formed by a vapor deposition method, a sputtering method, an ion plating method, screen printing, or the like. The porous moisture-sensitive member may be formed by application, screen printing, or the like. The second electrode may be formed by a vapor deposition method, a sputtering method, an ion plating method, screen printing, or the like.

Although embodiments have been described above, it will be understood that various changes in forms and details may be made without departing from the spirit and scope of the claims.

### <Respiration Sensing System>

The present disclosure further provides a respiration sensing system. The respiration sensing system refers to a system that senses respiration of a subject using a sensor, and the respiration sensing system in the present disclosure uses a humidity sensor. As the humidity sensor, the humidity sensor in the present disclosure described above is preferably used.

Since the respiration sensing system in the present disclosure uses a humidity sensor, it is less susceptible to vibration, and is advantageous as compared with a sensor that is susceptible to vibration, such as a millimeter-wave sensor or a sensor including a camera. Furthermore, since a humidity sensor including the porous moisture-sensitive member in the present disclosure exhibits good sensitivity and a favorable response speed, a respiration sensing system using a humidity sensor including the porous moisture-sensitive member in the present disclosure is less susceptible to wind, and can be favorably used also in a space having an air conditioning facility.

Although the direct measurement data obtained from the respiration sensing result of the respiration sensing system in the present disclosure is a function of the time variable, it can be converted into a function of the frequency variable by using the Fourier transform, and the respiration rate can be specified from the frequency of a peak of the function. The Fourier transform method is a method well known to those skilled in the art, and conditions thereof can be appropriately determined by any person skilled in the art. For example, the sampling interval is 50 ms, and the number of samples is 1024.

In the present disclosure, the exhalation source is the mouth or the nostrils of the subject.

In the present disclosure, a “front direction”, a “back direction”, a “leftward or rightward direction”, a “upward or downward direction”, and the like used directly or indirectly are directions based on a subject. Therefore, usually, the front direction is the travelling direction or the line-of-sight direction of the subject. In one embodiment, the vertically upward direction may correspond to the “upward direction”, and its opposite direction may correspond to the “downward direction”.

### [Humidity Sensor]

The respiration sensing system in the present disclosure preferably uses a humidity sensor using the porous moisture-sensitive member described above.

The sum of the response time and the recovery time of the humidity sensor used in the respiration sensing system of the present disclosure may be 0 seconds or more, 0.1 seconds or more, 0.5 seconds or more, or 1 second or more. The sum of the response time and the recovery time of the humidity sensor used in the respiration sensing system in the present disclosure may be 25 seconds or less, 20 seconds or less, 15 seconds or less, 10 seconds or less, 8 seconds or less, 5 seconds or less, 3 seconds or less, or 2 seconds or less, and is preferably 10 seconds or less, more preferably 5 seconds or less. The smaller the sum of the response time and the recovery time (the higher the responsiveness), the more accurately respiration can be measured. The method of measuring the response time and the recovery time is as described in EXAMPLES.

The sensitivity of the humidity sensor to be used in the respiration sensing system in the present disclosure may be 10 or more, 20 or more, 30 or more, 50 or more, 100 or more, 200 or more, 300 or more, or 500 or more, and is preferably 30 or more. The humidity sensor to be used in the respiration sensing system of the present disclosure may have a sensitivity of 3000 or less, 2000 or less, 1000 or less, 500 or less, or 250 or less. The method of measuring the sensitivity is as described in EXAMPLES.

### [Humidity Sensor Installation Region]

In the respiration sensing system of the present disclosure, the humidity sensor is disposed in a specific region in order to suitably sense respiration. The region in which the humidity sensor is disposed may mean a region in which a part or the entire of each humidity sensor is disposed, and may mean, for example, a region in which the center of gravity of the humidity sensor is disposed.

Examples of the place where the sensor is installed include a steering wheel of a vehicle, a seat belt, a necklace, a collar and a harness of a pet, a personal computer, a keyboard, a mouse, a television, a game controller, a wristwatch, a smartwatch, a mobile phone, a smartphone, a chest, an arm, a neck, an abdomen, a waist, a bed, and a pillow. As the installation place, a place where the position with respect to a subject is not necessarily fixed, such as a wristwatch, may be chosen. The subject may actively blow against the humidity sensor and measure respiration.

### (Sphere Region)

The region where the humidity sensor is disposed may be within a sphere region. Here, the sphere is centered at the exhalation source of a subject.

The radius of the sphere may be 0 cm or more, 3 cm or more, 5 cm or more, 10 cm or more, 15 cm or more, 20 cm or more, 25 cm or more, or 25 m or more. The radius of the sphere may be 150 cm or less, 140 cm or less, 120 cm or less, 100 cm or less, 80 cm or less, 70 cm or less, 60 cm or less, 40 cm or less, or 20 cm or less, and is preferably 100 cm or less, and particularly 70 cm or less. When a radius within the above range is chosen, good measurement results are easily obtained.

The region where the humidity sensor is disposed may be a region excluding a ¼ back upper region of the sphere. That is, the region where the humidity sensor is disposed may be a front upper side, a front lower side, or a back lower side of the sphere. When the region specified above is chosen as the region where the humidity sensor is disposed, good measurement results are easily obtained.

The linear distance between the humidity sensor installed within the sphere region and the exhalation source may be 0 cm or more, 3 cm or more, 5 cm or more, 7 cm or more, 10 cm or more, 15 cm or more, or 20 cm or more, and is preferably 5 cm or more or 10 cm or more from the viewpoint of selectively sensing a specific exhalation source.

### (Circular Cone A Region)

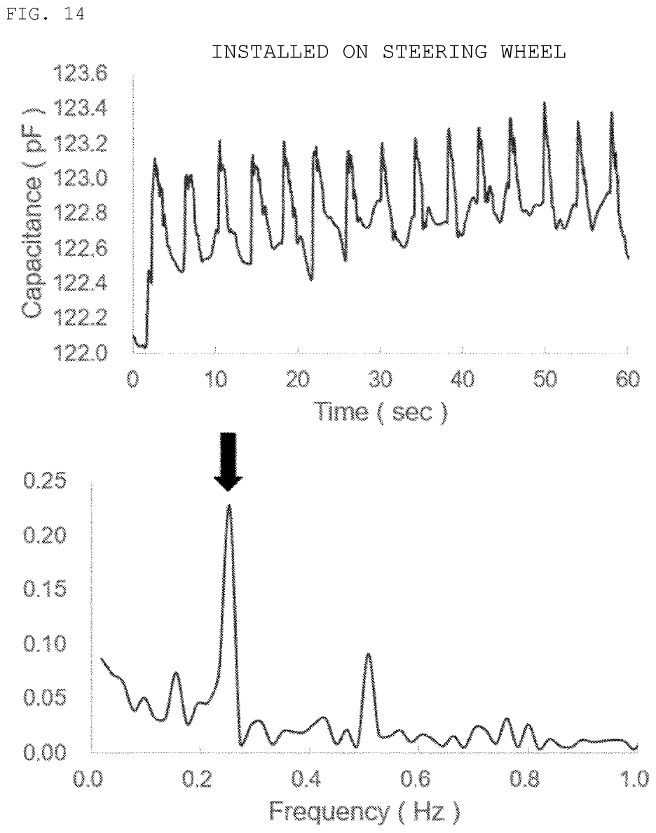

The region where the humidity sensor is disposed may be a circular cone A region. The circular cone A has an apex as an exhalation source of a subject and a rotation axis as a straight line extending from the exhalation source in a front direction of the subject. FIG. 11 is a schematic diagram of a circular cone A region in the respiration sensing system. The region within the circular cone 6 region in FIG. 11, preferably within the colored truncated circular cone region, may be a region in which the humidity sensor is disposed. Here, the height of the uncolored circular cone located at the top part of the circular cone 6 may be the minimum linear distance between the humidity sensor and the exhalation source.

A cone angle defined by the vertex angle of an isosceles triangle formed when the circular cone A is cut along a plane passing the rotation axis may be 0° or more, 3° or more, 5° or more, 8° or more, or 10° or more. The cone angle defined by the vertex angle of an isosceles triangle formed when the circular cone A is cut along a plane passing the rotation axis may be 90° or less, 600 or less, 40° or less, 35° or less, 30° or less, 25° or less, 20°, or 15° or less, and is preferably 40° or less. When the above range is chosen as the cone angle, good measurement results are easily obtained.

In the circular cone A, the cone height defined by the linear length from the center of the base to the apex may be 150 cm or less, 120 cm or less, 100 cm or less, 90 cm or less, 70 cm or less, 50 cm or less, 30 cm or less, or 20 cm or less, and is preferably 100 cm or less, particularly 70 cm or less. When the above range is chosen as the cone height, good measurement results are easily obtained.

The linear distance between the humidity sensor disposed within the circular cone A region and the exhalation source may be 0 cm or more, 3 cm or more, 5 cm or more, 7 cm or more, or 10 cm or more, and is preferably 5 cm or more. When the above range is chosen as the linear distance, good measurement results are easily obtained. When the humidity sensor is disposed within the circular cone A region, through providing a certain distance between the humidity sensor and the exhalation source, the respiration sensing system can more selectively sense an oral respiration component.

In a case where the region where the humidity sensor is disposed is the circular cone A, the exhalation source may be the mouth or the nostrils of the subject, and preferably may be the mouth of the subject. When the region where the humidity sensor is disposed is the circular cone A, the respiration sensing system is suitable for sensing an oral respiration component. Examples of a suitable installation place in a case where the region where the humidity sensor is disposed is the circular cone A region include a handle, a personal computer, and a television.

### (Circular Cone B Region)

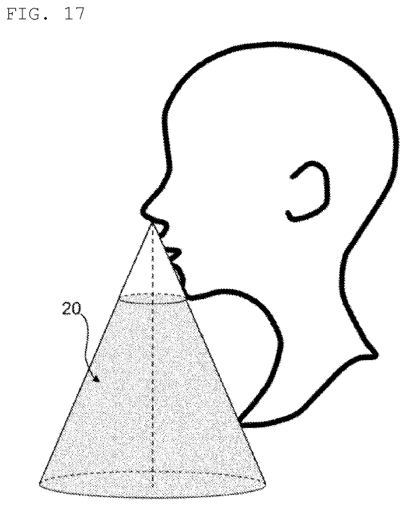

The region where the humidity sensor is disposed may be a circular cone B region. The circular cone B has an apex as the exhalation source and a rotation axis as a straight line extending downward by 30° and rightward or leftward by 30° with respect to a back direction from the exhalation source. FIG. 15 is a schematic diagram of a circular cone B region of a humidity sensor in a respiration sensing system. The region within the circular cone 14 or 14′ region in FIG. 15, preferably a colored truncated circular cone region, may be a region in which the humidity sensor is disposed. Here, the height of the uncolored circular cone located at the top part of the circular cone 14 or 14′ may be the minimum linear distance between the humidity sensor and the exhalation source.

A cone angle defined by the vertex angle of an isosceles triangle formed when the circular cone B is cut along a plane passing the rotation axis may be 0° or more, 3° or more, 5° or more, 8° or more, or 10° or more. The cone angle defined by the vertex angle of an isosceles triangle formed when the circular cone B is cut along a plane passing the rotation axis may be 90° or less, 800 or less, 600 or less, 40° or less, 35° or less, 30° or less, 250 or less, 20°, or 15° or less, and is preferably 60° or less. When the above range is chosen as the cone angle, good measurement results are easily obtained.

In the circular cone B, the cone height defined by the linear length from the center of the base to the apex may be 150 cm or less, 120 cm or less, 100 cm or less, 90 cm or less, 70 cm or less, 50 cm or less, 30 cm or less, 20 cm or less, or 10 cm or less, and is preferably 30 cm or less. When the above range is chosen as the cone height, good measurement results are easily obtained.

The linear distance between the humidity sensor disposed within the circular cone B region and the exhalation source may be 0 cm or more, 3 cm or more, 5 cm or more, 7 cm or more, or 10 cm or more, and is preferably 5 cm or more. When the above range is chosen as the linear distance, good measurement results are easily obtained.

In a case where the region where the humidity sensor is disposed is the circular cone B region, the exhalation source may be the mouth or the nostril of the subject. When the humidity sensor is disposed within the circular cone B region, the respiration sensing system is suitable for sensing an oral respiration component and/or a nasal respiration component. Examples of a suitable installation place in a case where the region where the humidity sensor is disposed is the circular cone B region include a necklace, a collar and a harness for a pet, and a neck.

### (Circular Cone C Region)

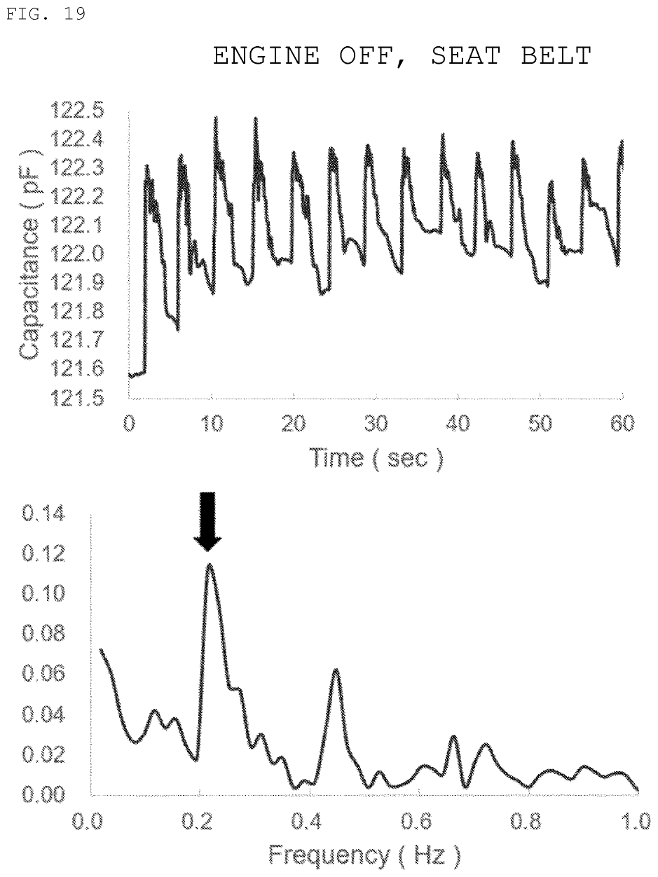

The region where the humidity sensor is disposed may be a circular cone C region. The circular cone C has an apex as the exhalation source and a rotation axis as a straight line extending from the exhalation source in a downward direction of the subject. FIG. 17 is a schematic diagram of a circular cone C region of a humidity sensor in a respiration sensing system. The region within the circular cone 20 region in FIG. 17, preferably within the colored truncated circular cone region, may be a region in which the humidity sensor is disposed. Here, the height of the uncolored circular cone located at the top part of the circular cone 20 may be the minimum linear distance between the humidity sensor and the exhalation source.

A cone angle defined by the vertex angle of an isosceles triangle formed when the circular cone C is cut along a plane passing the rotation axis may be 0° or more, 3° or more, 5° or more, 8° or more, or 10° or more. The cone angle defined by the vertex angle of an isosceles triangle formed when the circular cone C is cut along a plane passing the rotation axis may be 90° or less, 800 or less, 600 or less, 40° or less, 35° or less, 30° or less, 25° or less, 20°, or 15° or less, and is preferably 60° or less. When the above range is chosen as the cone angle, good measurement results are easily obtained.

In the circular cone C, the cone height defined by the linear length from the center of the base to the apex may be 150 cm or less, 120 cm or less, 100 cm or less, 90 cm or less, 70 cm or less, 50 cm or less, 30 cm or less, or 20 cm or less, and is preferably 70 cm or less. When the above range is chosen as the cone height, good measurement results are easily obtained.

The linear distance between the humidity sensor disposed within the circular cone C region and the exhalation source may be 0 cm or more, 3 cm or more, 5 cm or more, 7 cm or more, 10 cm or more, 15 cm or more, or 20 cm or more, and is preferably 5 cm or more, more preferably 10 cm or more. When the above range is chosen as the linear distance, good measurement results are easily obtained. When the humidity sensor is disposed within the circular cone C region, through providing a certain distance between the humidity sensor and the exhalation source, the respiration sensing system can more selectively sense a nasal respiration component.

In a case where the region where the humidity sensor is disposed is the circular cone C, the exhalation source may be the mouth or the nostrils of the subject, and preferably may be the nostrils of the subject. When the humidity sensor is disposed within the circular cone C region, the respiration sensing system is suitable for sensing a nasal respiration component. Examples of suitable installation places in the case where the region where the humidity sensor is disposed is the circular cone C region include a seat belt, a neck, an abdomen, and a waist.

### (Combination of a Plurality of Regions)

In the above description, the sphere region, the circular cone A region, the circular cone B region, and the circular cone C region are recited as examples of an installation place of the humidity sensor, but the installation place is not limited to them. In addition, it is also possible to install one or a plurality of sensors in one or a plurality of regions among the sphere region, the circular cone A region, the circular cone B region, and the circular cone C region. Examples of the combination include a combination of a circular cone A region and a circular cone B region, a combination of a circular cone A region and a circular cone C region, a combination of a circular cone B region and a circular cone C region, and a combination of a circular cone A region, a circular cone B region, and a circular cone C region. For example, since the circular cone A region is suitable for detecting oral respiration and the circular cone C region is suitable for detecting nasal respiration, an oral respiration component and a nasal respiration component may be separately measured by combining the circular cone A region and the circular cone C region. As described above, by separately measuring the oral respiration component and the nasal respiration component, it is possible to distinguish them and know which each person mainly does oral respiration or nasal respiration. The nose has a role of a filter for removing bacteria and viruses in the air, and humidified and warmed air is taken into the lungs by passing through the nose. Therefore, it is generally said that nasal respiration is better respiration. Therefore, it is also possible to correct the respiration to good respiration through knowing which of the oral respiration and the nasal respiration is performed.

### [Control Unit]

The respiration sensing system of the present disclosure may include a control unit that responds to a respiration sensing result.

The control unit can control a device. Here, the device is a device directly or indirectly connected to the humidity sensor. Examples of the device include, but are not limited to, automobiles, air conditioning devices, medical devices, and other industrial devices or home electric appliances.

Examples of the control of a device include temporary stop/restart of the function of the device, change of the function of the device, change of the strength of the function of the device, control of power ON/OFF of the device, and generation of a warning signal (display of warning, generation of warning sound, or generation of vibration alarm). For example, in a case where the device is an automobile, examples of control of the device include generation of a warning sound, braking, slowdown, direction change, and execution of automatic driving. For example, when the device is an air conditioning device, examples of control of the device include adjustment of comfortability (automatic adjustment) by adjustment of ambient temperature, adjustment of ambient humidity, adjustment of air volume, change of wind direction, or the like.

The respiration sensing system of the present disclosure can be used to adjust a surrounding environment (temperature, humidity, etc.) of a subject. The control unit can improve the comfortability of the subject by controlling the surrounding environment of the subject by an air conditioning facility based on the respiration sensing result and, as necessary, the information of the environment surrounding the subject. For example, a humidity sensor is installed on a seat belt of each seat to constantly measure the temperature and humidity around each seat, and the air conditioner is controlled in combination with HVAC when the condition around the seat becomes uncomfortable, whereby the comfortability in the vehicle can be further improved.

By using the respiration sensing system of the present disclosure, it is possible to sense a condition of an occupant or a driver by using information on respiration sensed in a vehicle (for example, a steering wheel or a seat belt). In recent years, accidents caused by deterioration in health condition of an occupant or a driver, such as deterioration in physical condition or sudden death of a driver during driving or leaving of a child, have become a problem. However, when the health condition deteriorates, the respiratory state also changes. Therefore, constantly monitoring the respiration using the respiration sensing system of the present disclosure makes it possible to know the condition of the occupant or the driver, and perform control such as making a warning sound as necessary or safely parking a car on a road shoulder.

### [Sensing of Second Vital Sign]

The respiration sensing system in the present disclosure may further include a sensor that measures second vibration data including a second vital sign other than respiration.

Examples of the sensor that measures the second vibration data including the second vital sign other than respiration include an optical sensors (visible light sensor, infrared sensor, etc.), a sound sensor, an electrical conductivity sensor, a potential sensor, a pressure sensor, and a heat sensor. The sensor that measures the second vibration data including the second vital sign other than respiration may be a sensor that is affected by vibration due to respiration, for example, an optical sensor.

Examples of the second vital sign other than respiration include pulse, heart rate, body temperature, and blood pressure.

The second vital sign can be sensed by subtracting the first vibration data based on the respiration sensing result from the second vibration data. As an example, a millimeter-wave sensor will be described as an example of the sensor that measures the second vibration data including the second vital sign. The way of thinking is the same for other sensors. Millimeter-wave sensors are known to be able to measure heart rate in addition to respiration. Respiration components include not only a fundamental wave that is an actual respiratory rate but also a harmonic component. In general, a heart rate is faster than a respiration rate. Therefore, a harmonic component of respiration also becomes a noise source when the heart rate is intended to accurately measure (see the following formula 1). On the other hand, since vital signs other than respiration cannot be measured by the respiration sensing using a humidity sensor, the response of the humidity sensor includes a fundamental wave and a harmonic component of respiration. By utilizing such a difference in sensors, the heart rate measured with a millimeter-wave sensor can be more accurately measured. That is, frequency components derived from a fundamental wave and a harmonic of respiration can be specified by using a humidity sensor. Since components derived from respiration are included in the same frequency also in the millimeter-wave sensor, it is possible to fully eliminate the components derived from respiration by deleting these components, and the remaining components are only heart rate components.

In recent years, sensing driver's condition has attracted attention, and sensing vital signs using a camera or a millimeter-wave sensor has attracted attention. However, since these measurement methods sense the displacement of the body due to respiration or heartbeat, there is a problem that there is an influence of vibration, and there is a problem that it is difficult to accurately measure. On the other hand, since a humidity sensor is not affected by vibration, respiration can be accurately measured therewith. Therefore, also when there is vibration, vital signs can be accurately measured by subtracting the respiration component determined with a humidity sensor from the components measured with a vital sign sensor as described above.

### EXAMPLES

The present disclosure will be described in detail below with reference to Examples, but the present disclosure is not limited only to Examples.

### <Evaluation Method>

Evaluation of a humidity sensor produced is as follows.

A capacitance change of the humidity sensor when humidity was changed was measured using a circuit FDC2214EVM (manufactured by Texas Instruments Inc.). Wet air produced with a bubbler is caused to flow at a flow rate of 1 SLM through a pipe having an inner diameter of 4 mm to a humidity sensor stabilized under a humidity atmosphere of 50% RH. At this time, the distance between the humidity sensor and the pipe was set to 10 mm. An existing humidity sensor SHT31 (manufactured by Sensirion AG) was also measured at the same time, and the ratio (fF/% RH) of the change in the capacitance display value of the FDC2214EVM circuit to the change in the humidity display value of this sensor was defined as the sensitivity of the humidity sensor. As the values, a value at the time of the largest change due to humidity change was used.

The responsiveness of the sensor is evaluated on the basis of the response time and the recovery time. The response time is a time taken until reaching 90% of the value when fully responding to moist air from baseline (commonly referred to as t₉₀), and the recovery time is a time taken until returning to 10% of the amount of change from the value when fully responding to moist air (commonly referred to as t₁₀).

### <Production and Performance Evaluation of Humidity Sensor>

### Example 1

A case where polyamideimide (PAI) was used as a base material of a moisture-sensitive film and Ni was used as an inorganic filler will be described. As the PAI, a varnish in which a PAI raw material was dissolved in N-methyl-2-pyrrolidone (NMP) and tetraglyme (TEGM) was used. The Ni filler used had an average particle size of about 300 nm. These raw materials were weighed such that the amount of Ni was 0 to 50% by volume, and mixed with a Hoover Muller such that the Ni filler was uniformly dispersed in the varnish. The varnish thus prepared was applied by screen printing to a 200 nm-thick NiCr lower electrode formed on a Kapton base plate by a sputtering method, and then dried in air at 150° C. for 5 minutes, forming a moisture-sensitive film in which the Ni filler was dispersed in a porous polyamideimide base material. Thereafter, an upper electrode having a thickness of 70 nm was formed by a sputtering method.

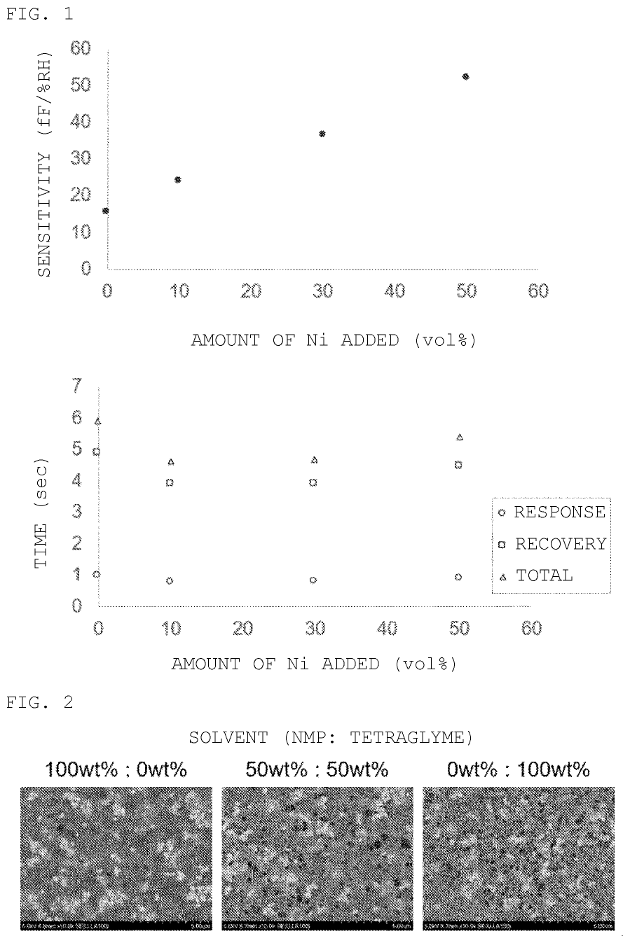

The sensitivity and responsiveness of the humidity sensor produced are shown in FIG. 1. It can be confirmed from FIG. 1 that the sensitivity is improved in all cases where Ni is added as compared with the case where Ni is not added. Further, also regarding responsiveness, it can be seen that better characteristics can be obtained in the case where Ni is added. These results show that the sensor characteristics are improved in sensitivity and responsiveness by dispersing the Ni filler in the porous PAI base material. In the case of the existing product, the response time was 4.1 seconds, the recovery time was 32.1 seconds, and the total time was 36.2 seconds. It is understood that since in the existing product a resin material that is not porous is used as a moisture-sensitive film, the responsiveness is overwhelmingly poor.

### Example 2

As an example, a description will be given using a result obtained when the amount of the solvent at the time of forming a moisture-sensitive film was varied. A 50% by weight solvent was added to the PAI varnish used in Example 1. The solvents added were designed to have ratios of NMP and TEGM was 100:0, 50:50, and 0:100, respectively. In addition, CaCu₃Ti₄O₂(CCTO) was used in place of Ni as an inorganic filler, and added in an amount of 50% by volume with respect to the PAI. A sensor was manufactured and evaluated in the same manner as in Example 1 except for these points.

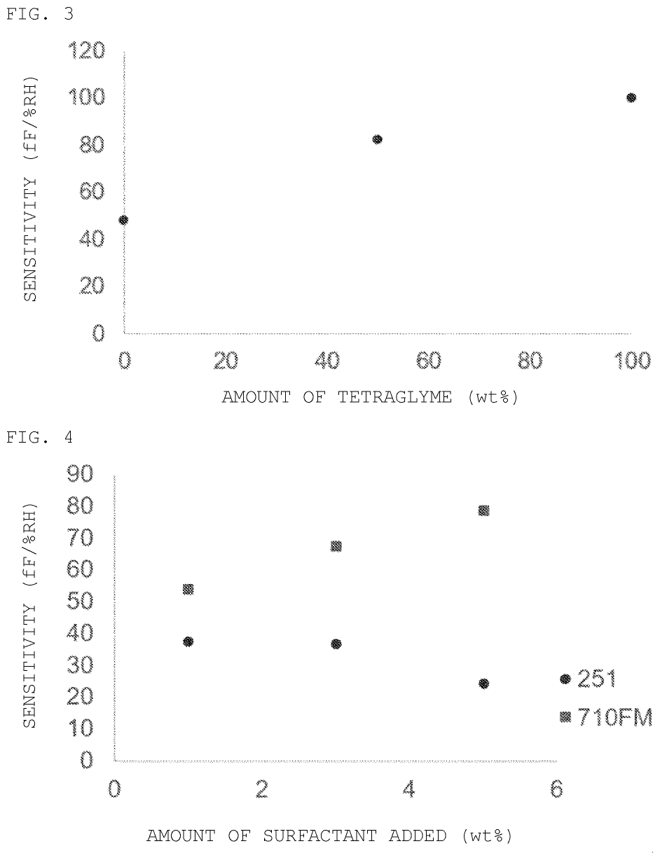

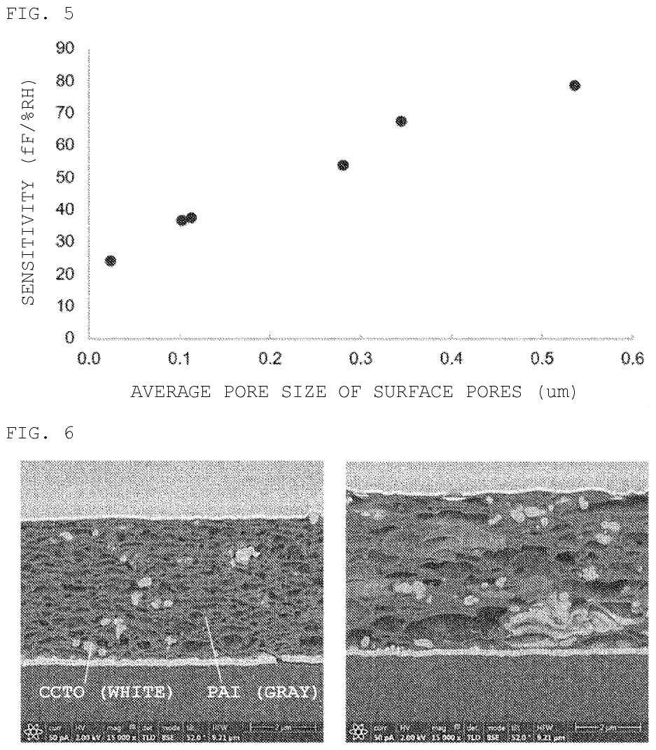

A surface SEM image of the humidity sensor produced is shown in FIG. 2. It can be seen from FIG. 2 that pores are formed up to the film surface as the amount of TEGM added increases. The sensitivity of these humidity sensors is shown in FIG. 3.

It can be seen from FIG. 2 that the density of surface pores increases as the amount of TEGM increases. It can be seen from FIG. 3 that the sensitivity is improved as the amount of TEGM is increased. From these results, it can be said that the sensitivity is improved as a result of an increase of the density of surface pores.

### Example 3

As an example, a description will be given using a result obtained when a surfactant is added. As a surfactant, FTERGENT 251, which is a hydrophilic surfactant or FTERGENT 710FM, which is a hydrophobic surfactant (both manufactured by NEOS Co., Ltd.) was used. In addition, CaCu₃Ti₄O₂(CCTO) was used in place of Ni as an inorganic filler, and added in an amount of 50% by volume with respect to the PAI. The surfactants were added at 1% by weight, 3% by weight, and 5% by weight with respect to CCTO. A sensor was manufactured and evaluated in the same manner as in Example 1 except for these points.

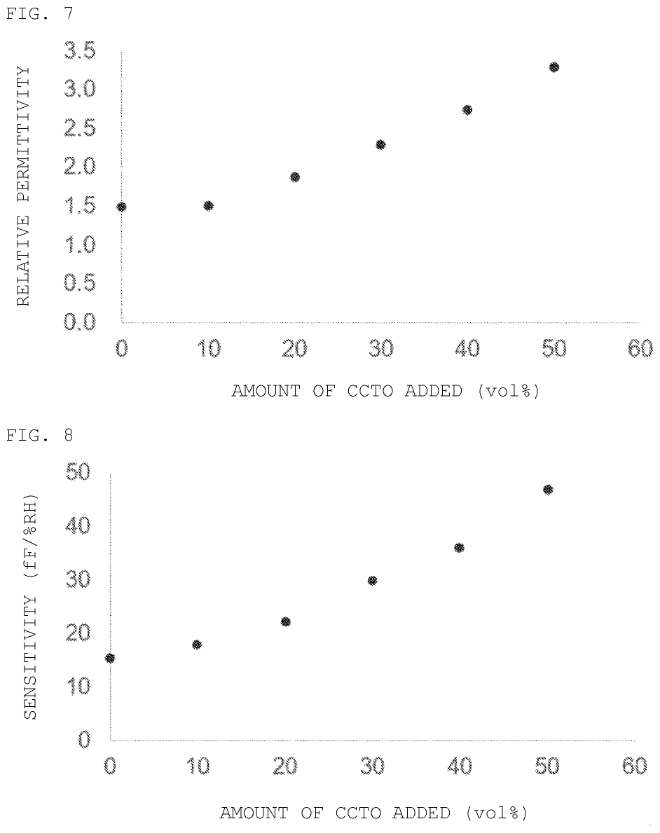

The sensitivity of the humidity sensor produced is shown in FIG. 4. From FIG. 4, in the case of FTERGENT 710FM, the sensitivity tended to be improved as the addition amount is increased.

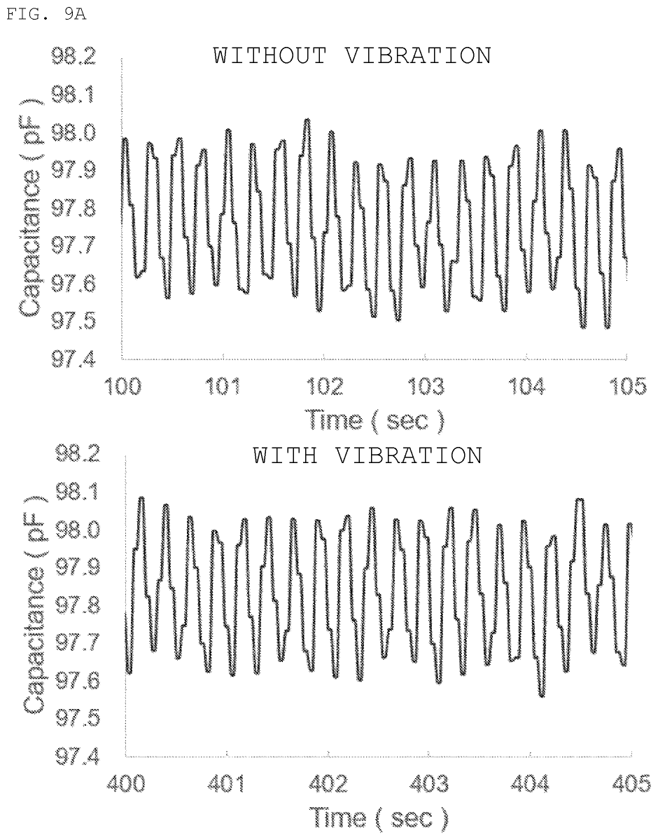

For the humidity sensors when 1% by weight, 3% by weight, or 5% by weight of each surfactant was added, surface SEM observation was performed and the average pore size of surface pores was calculated. Regarding the average pore size of surface pores, the longest size of each surface pore observed was regarded as the pore size of each surface pore, the pore sizes of all the surface pores existing in the visual field were measured, then the operation of moving the visual field and measuring pore sizes again was repeated, and the average value for 100 or more surface pores was taken as the average pore size of the surface pores. The calculated average pore sizes of the surface pores are shown in Table 1 together with the results of sensitivity measurement. FIG. 5 shows a correlation between the average pore size of surface pores of a moisture-sensitive film and sensitivity of a humidity sensor.

TABLE 1

| | Average pore size | |
|---|---|---|
| | of surface pores | Sensitivity |
| Surfactant | (um) | (fF/% RH) |

| | | | |
|---|---|---|---|
| FTERGENT | 1 Wt % | 0.113 | 37.5 |
| 251 | 3 Wt % | 0.102 | 36.8 |
| | 5 Wt % | 0.024 | 24.3 |
| FTERGENT | 1 Wt % | 0.281 | 54.0 |
| 710FM | 3 Wt % | 0.344 | 67.5 |
| | 5 Wt % | 0.536 | 78.6 |

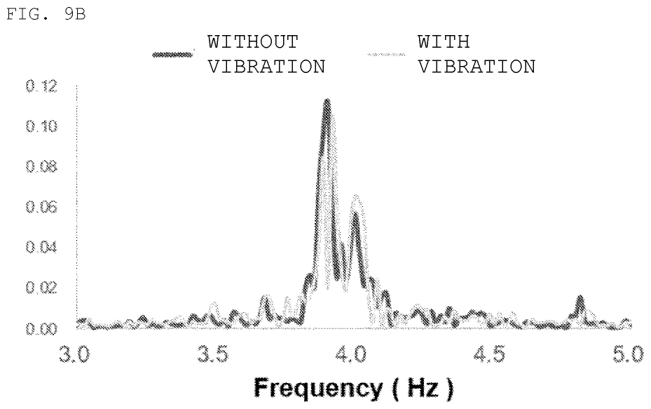

For the humidity sensors in the cases of adding 5% by weight of a surfactant, sectional SEM observation was performed. The results are shown in FIG. 6. As can be seen from FIG. 6, when FTERGENT 251 was added, most of the internal pores were small in diameter, and when FTERGENT 710FM was added, many internal pores are large in diameter. The BET specific surface area, sensitivity, and responsiveness of these elements are summarized in Table 2. It can be seen from Table 2 that when a surfactant (FTERGENT 710FM) is added, the sensitivity and responsiveness can be improved although the specific surface area is small.

TABLE 2

| | FTERGENT 251 | FTERGENT 710FM |
|---|---|---|
| Surfactant (5 wt %) | (Hydrophilic) | (Hydrophobic) |
| Specific surface area (m2/g) | 2.02 | 1.42 |
| Sensitivity (fF/% RH) | 24.3 | 78.6 |
| Response + recovery (sec) | 2.84 | 1.85 |
| | (Response: 0.60/ | (Response: 0.85/ |
| | Recovery: 2.24) | Recovery: 1.00) |

That is, from these results, it can be seen that the pore sizes and the densities of surface pores and internal pores of a moisture-sensitive film can be adjusted by adding a surfactant, and the sensitivity and responsiveness of a sensor can be improved.

### Example 4

As an example, a case where the added amount of CCTO was varied will be described. CCTO was added at 0 to 50% by volume with respect to PAI. A sensor was manufactured and evaluated in the same manner as in Example 1 except for this point.

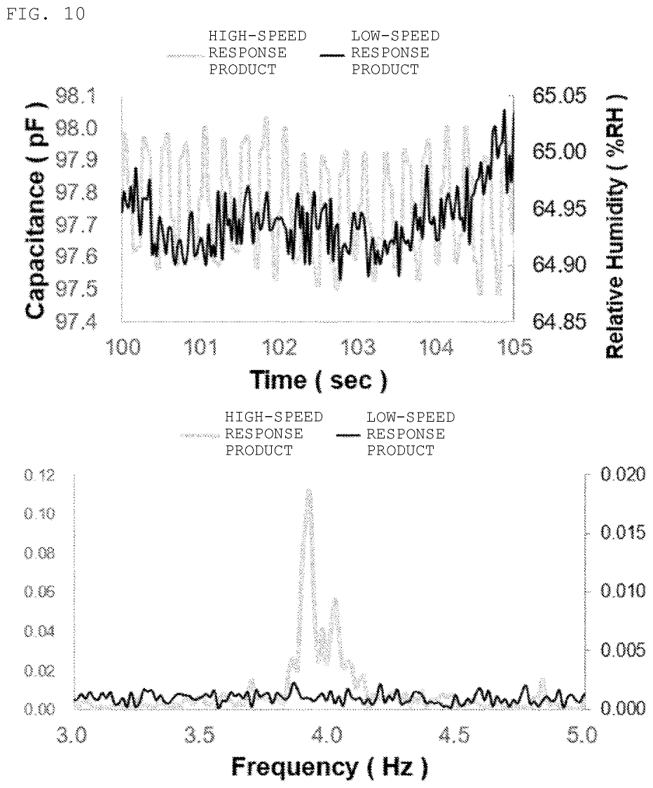

The dielectric constant of the moisture-sensitive film prepared is shown in FIG. 7. It can be confirmed from FIG. 7 that the dielectric constant increases as the added amount of CCTO increases. Furthermore, it can be seen from FIG. 8 that the sensitivity also increases as the added amount of CCTO increases, similarly to the dielectric constant. From these results, it can be seen that the sensitivity is improved as the dielectric constant of the moisture-sensitive film increases.

### <Performance Evaluation of Respiration Sensing System>

### Example 5

### (Humidity Sensor Used)

The responsiveness was measured for three types of humidity sensors using the porous moisture-sensitive member in the present disclosure (hereinafter, these sensors are referred to as high-speed response products (1), (2), and (3), respectively) and a commercial product (SHT31, manufactured by Sensirion AG; hereinafter, this is referred to as a low-speed response product).

The high-speed response products (1), (2), and (3) had response speeds of 1.9 seconds, 1.3 seconds, and 0.9 seconds, and recovery speeds of 4.9 seconds, 2.5 seconds, and 1.6 seconds, respectively. The low-speed response product had a response speed of 5.5 seconds and a recovery speed of 16.1 seconds.

### (Confirmation of Influence of Vibration)

First, in order to show that there is no influence of vibration in respiration measurement, the following experiment was performed. A sensor, an optical chopper, and a bubbler were all vibrated in a state where wet air passed through the bubbler at a frequency of 4 Hz hit the sensor using the optical chopper. The wet air that has passed through the bubbler corresponds to simulated respiration. This test was conducted under domestic transportation standard conditions (JIS Z 0232 standard, 5 to 200 Hz, Grms=0.59, random vibration, vertical direction) using a vibration tester.

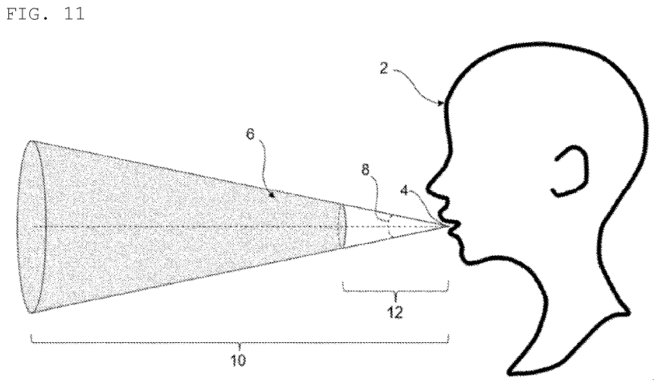

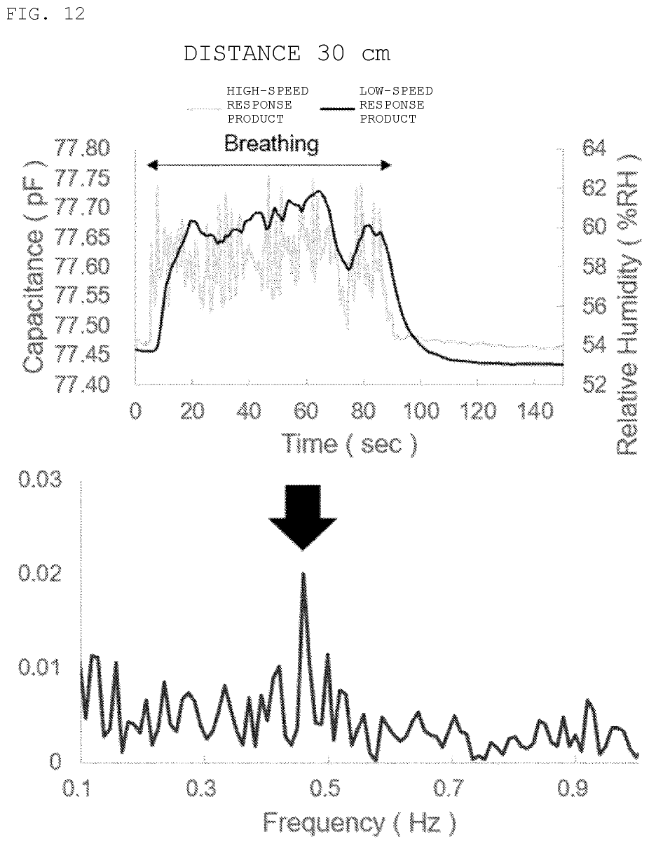

FIGS. 9A and 9B show the measurement data of the case without vibration (100 to 105 seconds after the start of measurement) and the case with vibration (400 to 405 seconds after the start of measurement) for the high-speed response product (2), and the results obtained by Fourier transforming those results. From FIGS. 9A and 9B, there was no difference in the measurement data between with and without vibration. In addition, since no difference in the Fourier transformed results between with and without vibration was observed, it can be seen that high-speed response products can measure a humidity change without being affected by vibration, that is, can measure respiration without being affected by vibration.

### (Influence of Difference in Responsiveness, and Measurement in Circular Cone A Region)

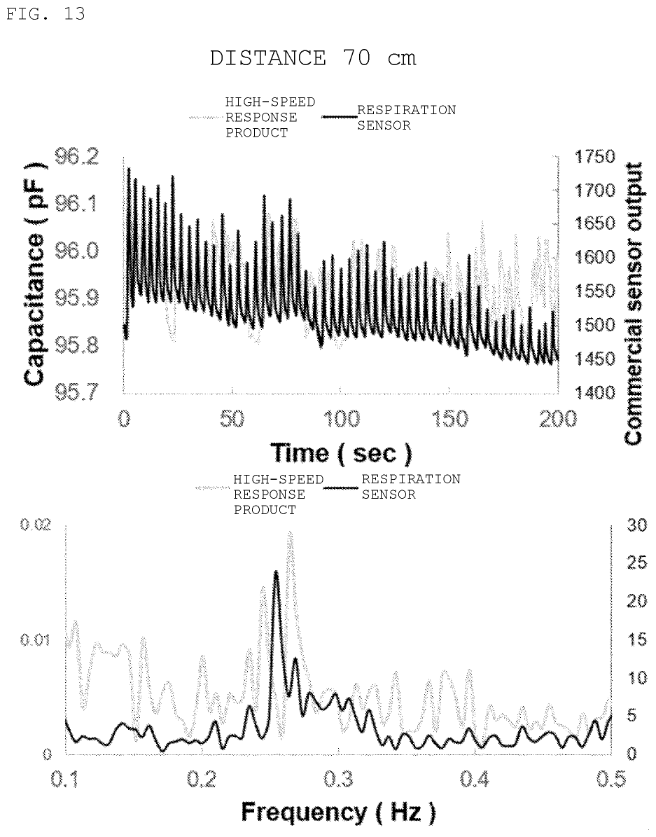

Comparison results (measurement data and Fourier transformed result) between the high-speed response product (2) and the low-speed response product without vibration are shown in FIG. 10. It can be seen from FIG. 10 that the low-speed response product cannot follow the frequency change of 4 Hz. This is considered to be because the response speed and the recovery speed are slow.

Then, a difference in response between the high-speed response product (1) and the low-speed response product was decided to be examined in actual respiration measurement. Each sensor was installed on the upper part of a PC screen in front of a subject such that the distance between the sensor and the mouth was approximately 30 cm, and the measurement was performed. At this time, the humidity sensor is disposed within the truncated circular cone region (colored region) in the circular cone A region as illustrated in FIG. 11. The measurement results and a result of Fourier transform thereof are shown in FIG. 12. From FIG. 12, a response due to respiration can be confirmed with the high-speed response product (1), and it can be seen from the Fourier transformed result that the respiration rate is about 27.5 times/min. On the other hand, a respiratory response could not be confirmed with the low-speed response product.

From these results, it can be seen that the response speed and recovery speed of a humidity sensor are very important in respiration measurement.

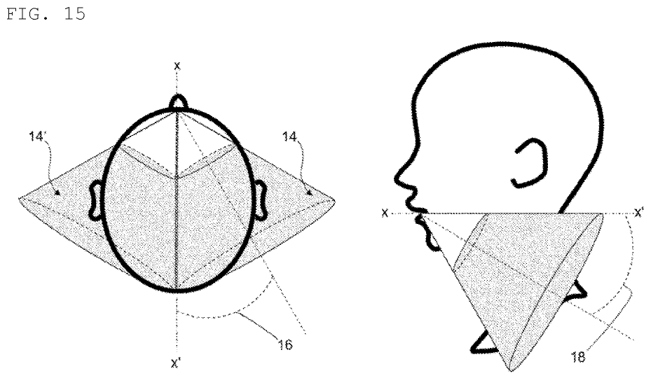

Here, in order to confirm that the response obtained in FIG. 12A is actually a response derived from respiration and to check how far at most the sensor and the mouth may be separated from each other to enable measurement, measurement was performed with the distance between the sensor and the mouth set to about 70 cm. At this time, an existing respiration sensor (YSSDA, manufactured by ATR-Promotions Inc.) was wound around the chest with a belt to be used as a reference of respiration. The results of the measurement using the high-speed response product (2) are shown in FIG. 13. As can be seen from FIG. 13, a response was observed in the high-speed response product (2) also with a distance of 70 cm, and a very good agreement was obtained when comparing the result of Fourier transform with the existing respiration sensor. This shows that respiration can be measured also when the sensor and the mouth are separated from each other by 70 cm.

In addition, in the measurement data of FIGS. 12 and 13, there is a part where no response is observed, and this is because the face moved during the measurement.

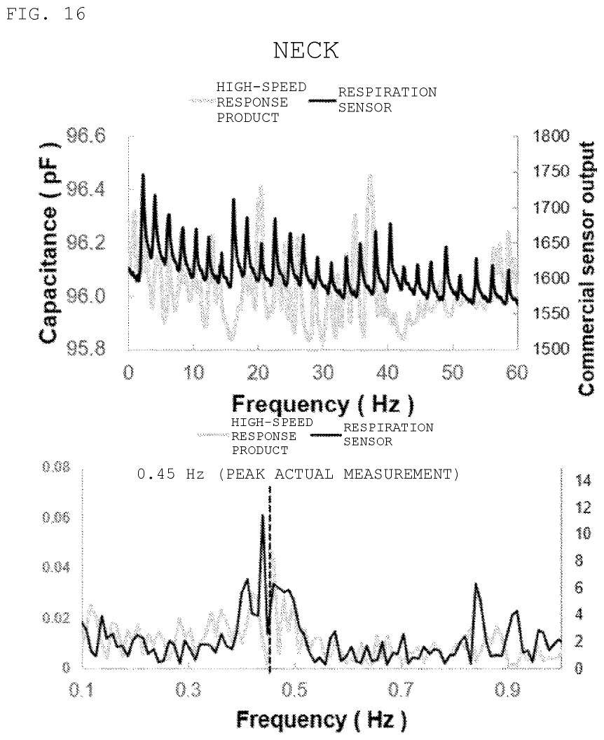

Similarly, the measurement data and a result of Fourier transform thereof in a case where the high-speed response product (3) was installed on a steering wheel of a car are shown in FIG. 14. It can be seen from FIG. 14 that respiration can be measured by installing a sensor at the position of a steering wheel.

### Example 6

### (Measurement in Circular Cone B Region)

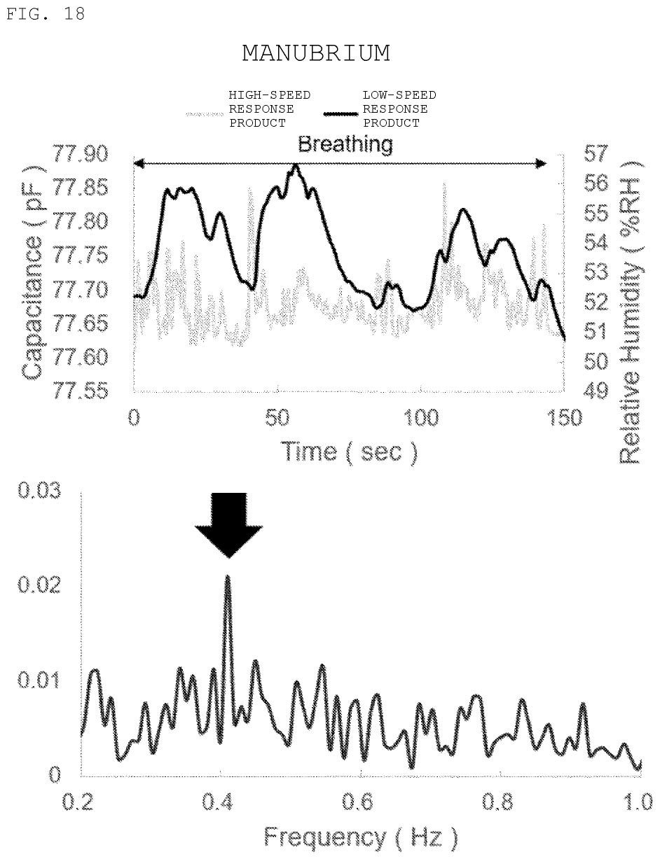

A case where the high-speed response product (2) was installed near the carotid artery of the neck will be described. At this time, the humidity sensor is disposed within the truncated circular cone region (colored region) in the circular cone B region as illustrated in FIG. 15. The existing respiration sensor described in Example 5 was used to confirm whether respiration could be measured. The measurement data and a result of Fourier transform thereof are shown in FIG. 16. It can be seen from FIG. 16 that very good agreement is obtained between the existing respiration sensor and the high-speed response product (2), and respiration can be measured by installing a humidity sensor at the neck. There is a time during which a response is not observed partly during the measurement, and this is because the face moved.

Since the sensor can be installed at this position to measure respiration, it can be seen that it is possible to constantly monitor respiration without being affected by vibration, for example, by installing the sensor on a necklace, or a collar or harness for a pet.

### Example 7

### (Measurement in Circular Cone C Region)

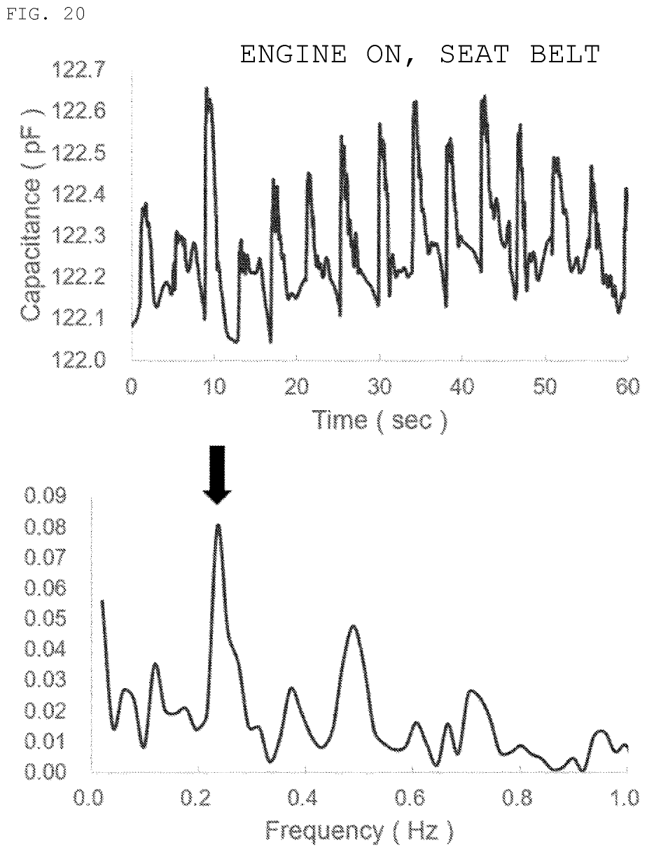

An example of respiration sensing in a case where the high-speed response product (1) and the low-response product were placed at the neck (manubrium) will be described. At this time, the humidity sensor is disposed within the truncated circular cone region (colored region) in the circular cone C region as illustrated in FIG. 17. The measurement data and a result of Fourier transform thereof are shown in FIG. 18. As can be seen from FIG. 18, while the low-speed response product cannot follow respiration, the response by respiration can be confirmed with the high-speed response product (1).

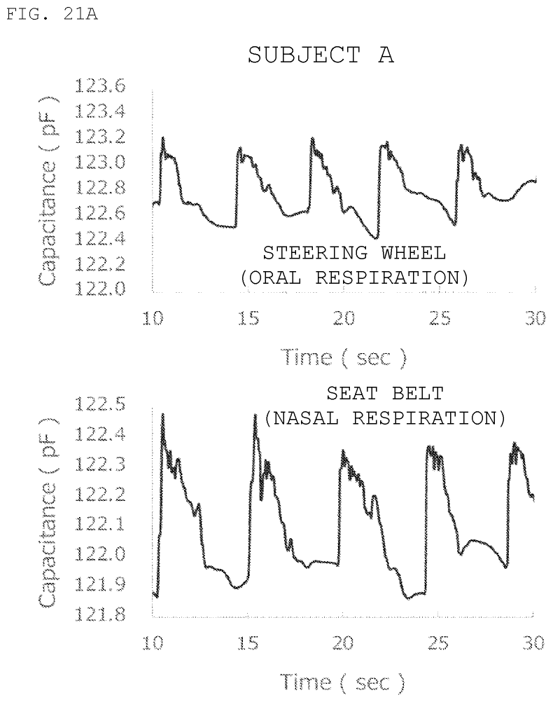

Next, a case where the high-speed response product (3) was installed on a seat belt (near the center of the body) of a car will be described. At this time, the humidity sensor is disposed within the truncated circular cone region (colored region) in the circular cone C region as illustrated in FIG. 17. The data measured in an engine OFF state and a result of Fourier transform thereof are shown in FIG. 19. It can be seen from FIG. 19 that the nasal respiration component can be measured at the position of the seat belt. Further, the data measured in an engine ON state and a result of Fourier transform thereof are shown in FIG. 20. It can be seen from FIG. 20 that also when there is vibration of a car, respiration can be measured as in the case of engine OFF.

### Example 8

### (Measurement in Circular Cone A Region and Circular Cone C Region)

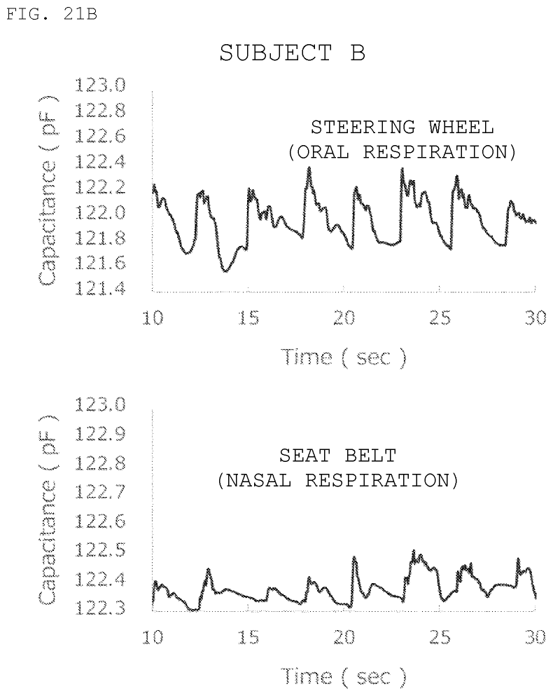

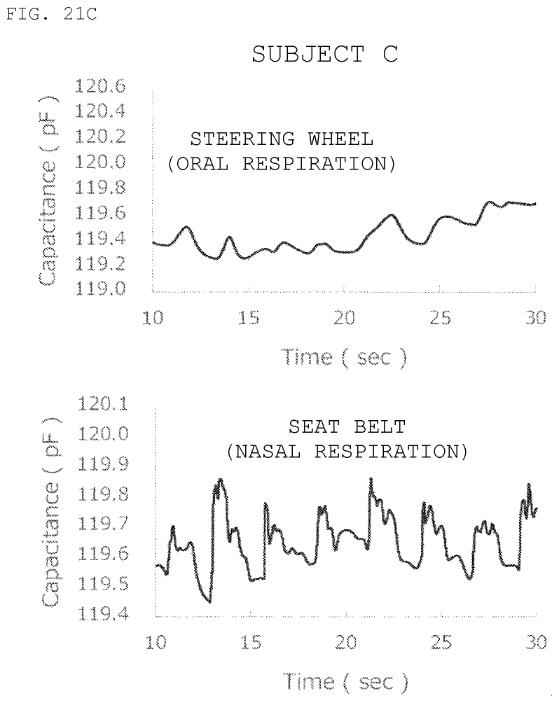

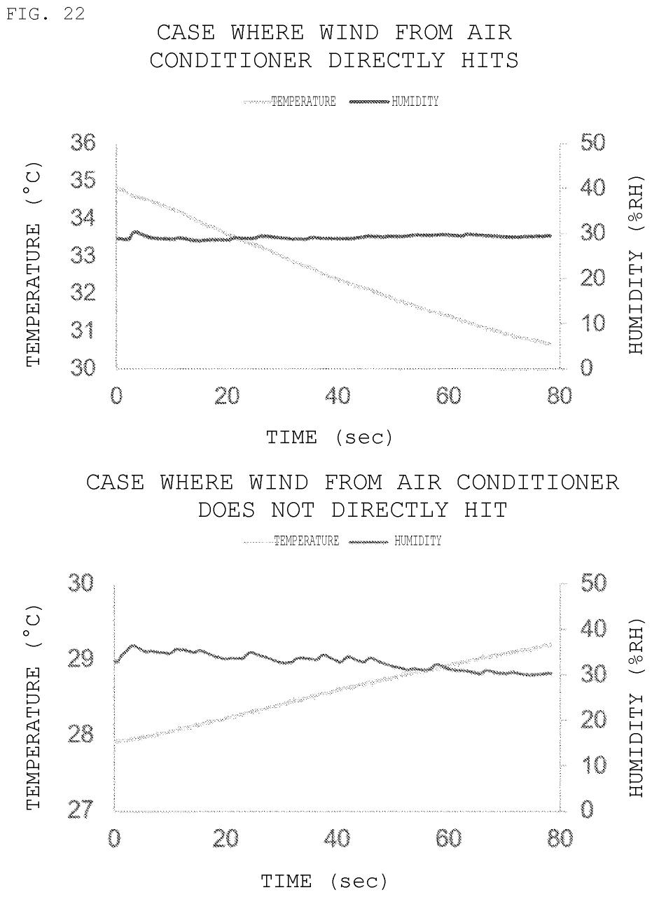

In Example 5, it was shown that the oral respiration component can be measured when the high-speed response product is installed on a steering wheel. Then, the measurement results at the steering wheel position and the seat belt position of three subjects are shown in FIGS. 21A to 21C (the results shown so far were obtained by the subject A). At this time, the humidity sensor is disposed both within the truncated circular cone region (colored region) in the circular cone A region as illustrated in FIG. 11 and within the truncated circular cone region (colored region) in the circular cone C region as illustrated in FIG. 17. In order to compare the measurement results of the three subjects, the absolute values of the sizes of the vertical axis and the horizontal axis are adjusted. It was found from FIGS. 21A to 21C that the response varies among the three subjects. The subject A has a large response to both oral respiration and nasal respiration, while the subject B has a large oral respiration but a small nasal respiration. Conversely, the subject C has small mouth respiration but large nasal respiration. As described above, it has been found that by separately measuring the oral respiration component and the nasal respiration component, it is possible to distinguish them and know which each person mainly does oral respiration or nasal respiration. The nose has a role of a filter for removing bacteria and viruses in the air, and humidified and warmed air is taken into the lungs by passing through the nose. Therefore, it is generally said that nasal respiration is better respiration. Therefore, it is also possible to correct the respiration to good respiration through knowing which of the oral respiration and the nasal respiration is performed.

Furthermore, it can be confirmed from FIGS. 21A to 21C that the subjects sharply respond in both cases of oral respiration and nasal respiration, and then slowly recover. This indicates that the form of respiration has been measured well. This is because the response and recovery of a humidity sensor are fast, so that the humidity change due to respiration can be accurately measured.

### Example 9

### (Combined Use of Air Conditioner)

In order to control an air conditioner of a car, a temperature and humidity sensor is currently installed around the air conditioner. However, since the sensor is far from a driver or an occupant and the temperature and humidity therearound is different from the temperature and humidity around the occupant or the driver, an uncomfortable state is often produced when the air conditioner is not functioning well.

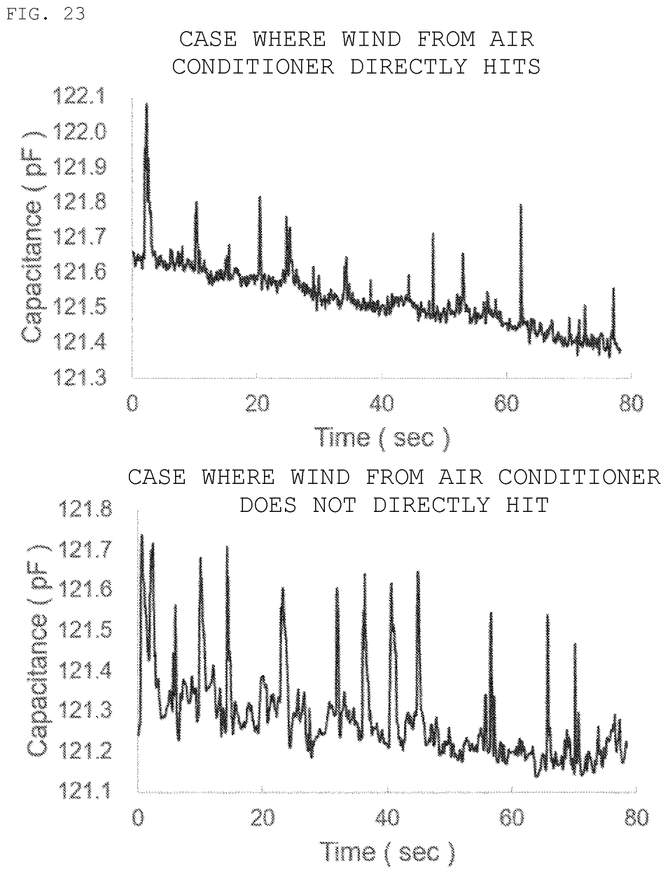

Then, a temperature and humidity sensor was installed at the seat belt position similarly to the experiment described in Example 3, and the temperature and humidity around a driver were measured. For the measurement, a temperature and humidity sensor (SHT31, manufactured by Sensirion AG, low-speed response product) mounted on a BLE module was used. Temperature and humidity changes were measured when the air conditioner was turned on with a middle fan speed. In addition, the case where the air conditioner directly hit the body and the case where the air conditioner did not hit the body with a constant strength were measured in this order. The results are shown in FIG. 22. It can be seen from FIG. 22 that the temperature gradually decreased as the wind from the air conditioner directly hit the body. On the other hand, it can be seen that the temperature gradually increased when the direction of the wind was adjusted such that the wind did not hit the body though the air conditioner had been turned on with the same strength. As an actual feeling, the driver felt cool when the wind from the air conditioner hit the body, but gradually felt hot when the air was made not to hit the body. Therefore, it can be seen that the temperature and humidity around the driver could be measured more accurately.

For example, the same sensor is installed on a seat belt of each seat to constantly measure the temperature and humidity around each seat, and the air conditioner is controlled in combination with HVAC when the condition around the seat becomes uncomfortable, whereby the comfortability in the vehicle can be further improved.

Although the temperature and humidity sensor equipped with a BLE module was used in FIG. 22, the high-speed response product described in Example 3 may be used for the display value of humidity. Since the output value of the high-speed response product used this time is a capacity, the capacity can be converted into a humidity by examining the relationship (calibration curve) between the capacity and the humidity in advance. At this time, a large slow variation of the baseline corresponds to a change in the humidity around the subject, whereas a small fast variation corresponds to a change due to respiration of the subject.

Incidentally, it is conceivable that when the wind from the air conditioner hits the humidity sensor, respiration cannot be measured. Then, at the same time as the experiment shown in FIG. 22, respiration measurement was performed at the seat belt position using the high-speed response product (3), and the data shown in FIG. 23 were obtained. In both the cases, a response considered to be caused by respiration is observed. However, the appearance is changed in both the cases as compared with the case of the air conditioner OFF shown in Example 5, and when there is direct wind, it seems that the influence is particularly large. However, when Fourier transform of these data was performed, a frequency component corresponding to the respiration rate was included, and the respiration rate was measurable even if the humidity around the subject was changed by turning on the air conditioner.

The porous moisture-sensitive member in the present disclosure and the humidity sensor using the porous moisture-sensitive member can be used for various applications such as home electric devices, industrial devices, and in-vehicle applications.

### DESCRIPTION OF REFERENCE SYMBOLS

2: Subject
4: Exhalation source
6: Circular cone A region
8: Cone angle
10: Cone height
12: Possible minimum distance between humidity sensor and exhalation source
14: Circular cone B region (right side)
14′: Circular cone B region (left side)
16: Angle of rotation axis of circular cone C with reference to back direction (60° to right)
18: Angle of rotation axis of circular cone C with reference to back direction (30° downward)
x: Front direction
x′: Back direction
20: Circular cone C region

### EXEMPLARY EMBODIMENTS

Exemplary embodiments of the present disclosure are as follows.

[Item 1] A porous moisture-sensitive member for a humidity sensor, the porous moisture-sensitive member comprising: a resin base material; and an inorganic filler, wherein the porous moisture-sensitive member contains surface pores and internal pores, and the surface pores have an average pore size of 0.1 μm or more.

[Item 2] The porous moisture-sensitive member according to Item 1, wherein the surface pores that have a pore size of 0.1 μm or more have a density of 1 pore/100 μm² or more.

[Item 3] The porous moisture-sensitive member according to Item 1 or 2, wherein the surface pores that have a pore size of 0.1 μm or more have a density of 50 pores/100 μm² or more.

[Item 4] The porous moisture-sensitive member according to any one of Items 1 to 3, wherein a pore section having a sectional size of 1 μm or more exists in a section of the porous moisture-sensitive member.

[Item 5] The porous moisture-sensitive member according to any one of Items 1 to 4, wherein a density of pore sections having a sectional size of 1 μm or more in a section of the porous moisture-sensitive member is 10 pore sections/100 μm² or more.

[Item 6] The porous moisture-sensitive member according to any one of Items 1 to 5, wherein the porous moisture-sensitive member has a specific surface area of 0.1 m²/g to 10 m²/g.

[Item 7] The porous moisture-sensitive member according to any one of Items 1 to 6, wherein the porous moisture-sensitive member has a specific surface area of 2 m²/g or less.

[Item 8] The porous moisture-sensitive member according to any one of Items 1 to 7, wherein the porous moisture-sensitive member has a porosity of 10% by volume to 90% by volume.

[Item 9] The porous moisture-sensitive member according to any one of Items 1 to 8, wherein the porous moisture-sensitive member has a relative permittivity of 1.6 or more.

[Item 10] The porous moisture-sensitive member according to any one of Items 1 to 9, wherein an amount of the resin base material is 10% by weight to 90% by weight with respect to the porous moisture-sensitive member, and an amount of the inorganic filler is 10% by weight to 90% by weight with respect to the porous moisture-sensitive member.

[Item 11] The porous moisture-sensitive member according to any one of Items 1 to 10, wherein the resin base material is at least one selected from the group consisting of aromatic polyimide, aromatic polyamideimide, aromatic polyamide, aromatic polyether, polyethylene terephthalate, cellulose acetate butyrate, polymethyl methacrylate, and vinyl crotonate.

[Item 12] The porous moisture-sensitive member according to any one of Items 1 to 11, wherein the inorganic filler comprises a high-dielectric material having a relative permittivity of 100 or more at 25° C. and 1 kHz.

[Item 13] The porous moisture-sensitive member according to any one of Items 1 to 12, wherein the inorganic filler comprises a metal-based filler.

[Item 14] The porous moisture-sensitive member according to any one of Items 1 to 13, wherein the porous moisture-sensitive member comprises a surfactant, and an amount of the surfactant is 0.1% by weight to 25% by weight with respect to the porous moisture-sensitive member.

[Item 15] The porous moisture-sensitive member according to any one of Items 1 to 14, wherein the porous moisture-sensitive member is intended for a capacitance change type humidity sensor.

[Item 16] A humidity sensor including: a first electrode; a second electrode; and the porous moisture-sensitive member according to any one of Items 1 to 15 provided between the first electrode and the second electrode.

[Item 17] The humidity sensor according to Item 16, further including a base plate, wherein the first electrode is provided on the base plate.

[Item 18] The humidity sensor according to Item 16 or 17, which is of a capacitance change type.

[Item 19] A respiration sensing system for sensing respiration of a subject, the respiration sensing system comprising: the humidity sensor according to any one of Items 16 to 18 within a sphere region defined by a sphere that has a center as an exhalation source of the subject and has a radius of 150 cm or less.

[Item 20] The respiration sensing system according to Item 19, wherein the humidity sensor is disposed in the region excluding a ¼ back upper region of the sphere.

[Item 21] The respiration sensing system according to Item 19 or 20, wherein the humidity sensor is disposed within a circular cone A region; the circular cone A has an apex as the exhalation source and a rotation axis as a straight line extending from the exhalation source in a front direction of the subject, a cone angle defined by a vertex angle of an isosceles triangle formed when the circular cone A is cut along a plane passing through the rotation axis is 40° or less, and a cone height defined by a straight line length from a base center to the apex is 100 cm or less.

[Item 22] The respiration sensing system according to Item 19 or 20, wherein the humidity sensor is disposed within a circular cone B region; the circular cone B has an apex as the exhalation source and a rotation axis as a straight line extending downward by 30° and rightward or leftward by 30° with respect to a back direction from the exhalation source, a cone angle defined by a vertex angle of an isosceles triangle formed when the circular cone B is cut along a plane passing through the rotation axis is 60° or less, and a cone height defined by a straight line length from a base center to the apex is 30 cm or less.

[Item 23] The respiration sensing system according to Item 21, wherein the exhalation source is the mouth of the subject, and the respiration sensing system senses an oral respiration component; or the respiration sensing system according to Item 22, wherein the exhalation source is the mouth and/or the nostrils of the subject and the respiration sensing system senses an oral respiration component and/or a nasal respiration component.

[Item 24] The respiration sensing system according to Item 19 or 20, wherein the humidity sensor is disposed within a circular cone C region; the circular cone C has an apex as the exhalation source and a rotation axis as a straight line extending from the exhalation source in a downward direction of the subject, a cone angle defined by a vertex angle of an isosceles triangle formed when the circular cone C is cut along a plane passing through the rotation axis is 60° or less, and a cone height defined by a straight line length from a base center to the apex is 70 cm or less.

[Item 25] The respiration sensing system according to Item 24, wherein the exhalation source is the nostrils of the subject and the respiration sensing system detects a nasal respiration component.

[Item 26] The respiration sensing system according to any one of Items 19 to 25, wherein a sum of a response time and a recovery time of the humidity sensor is 5 seconds or less.

[Item 27] The respiration sensing system according to any one of Items 19 to 26, including a control unit that responds to a respiration sensing result.

[Item 28] The respiration sensing system according to any one of Items 19 to 27, wherein the control unit controls an ambient environment of the subject by an air conditioning device on the basis of the respiration sensing result and, as necessary, other ambient environment information of the subject such as ambient temperature of the subject.

[Item 29] The respiration sensing system according to Item 28, wherein the control unit controls a device.

[Item 30] The respiration sensing system according to Item 29, wherein the device is an automobile.

[Item 31] The respiration sensing system according to any of Item 19 to 30, further including a sensor configured to measure second vibration data including a second vital sign other than respiration.

[Item 32] The respiration sensing system according to Item 31, wherein a second vital sign is sensed by subtracting first vibration data based on a respiration sensing result from the second vibration data.

[Item 33] The respiration sensing system according to any one of Items 19 to 32, wherein the humidity sensor is installed on a steering wheel of an automobile.

[Item 34] The respiration sensing system according to any one of Items 19 to 32, wherein the humidity sensor is installed on a personal computer or a personal computer-related device.

[Item 35] The respiration sensing system according to any one of Items 19 to 32, wherein the humidity sensor is installed on a neck.

[Item 36] The respiration sensing system according to any one of Items 19 to 32, wherein the humidity sensor is installed on a collar or harness for a pet (e.g., dog or cat).

[Item 37] The respiration sensing system according to any one of Items 19 to 32, wherein the humidity sensor is installed on a seat belt of an automobile.

[Item 38] The respiration sensing system according to any one of Items 19 to 37, wherein a linear distance between the humidity sensor and the exhalation source is 5 cm or more.

[Item 39] The respiration sensing system according to any one of Items 19 to 38, wherein a linear distance between the humidity sensor and the exhalation source is 10 cm or more.

1. A porous moisture-sensitive member for a humidity sensor, the porous moisture-sensitive member comprising:
a resin base material; and
an inorganic filler, wherein
the porous moisture-sensitive member contains surface pores and internal pores, and
the surface pores have an average pore size of 0.1 μm or more.

2. The porous moisture-sensitive member according to claim 1, wherein the surface pores have a density of 1 pore/100 μm² or more.

3. The porous moisture-sensitive member according to claim 1, wherein the surface pores have a density of 50 pores/100 μm² or more.

4. The porous moisture-sensitive member according to claim 1, wherein a pore section having a sectional size of 1 μm or more exists in a section of the porous moisture-sensitive member.

5. The porous moisture-sensitive member according to claim 1, wherein a density of pore sections having a sectional size of 1 μm or more in a section of the porous moisture-sensitive member is 10 pore sections/100 μm² or more.

6. The porous moisture-sensitive member according to claim 1, wherein the porous moisture-sensitive member has a specific surface area of 0.1 m²/g to 10 m²/g.

7. The porous moisture-sensitive member according to claim 1, wherein the porous moisture-sensitive member has a specific surface area of 2 m²/g or less.

8. The porous moisture-sensitive member according to claim 1, wherein the porous moisture-sensitive member has a porosity of 10% by volume to 90% by volume.

9. The porous moisture-sensitive member according to claim 1, wherein the porous moisture-sensitive member has a relative permittivity of 1.6 or more.

10. The porous moisture-sensitive member according to claim 1, wherein an amount of the resin base material is 10% by weight to 90% by weight with respect to the porous moisture-sensitive member, and
an amount of the inorganic filler is 10% by weight to 90% by weight with respect to the porous moisture-sensitive member.

11. The porous moisture-sensitive member according to claim 1, wherein the resin base material is at least one selected from the group consisting of aromatic polyimide, aromatic polyamideimide, aromatic polyamide, aromatic polyether, polyethylene terephthalate, cellulose acetate butyrate, polymethyl methacrylate, and vinyl crotonate.

12. The porous moisture-sensitive member according to claim 1, wherein the inorganic filler comprises a dielectric material having a relative permittivity of 100 or more at 25° C. and 1 kHz.

13. The porous moisture-sensitive member according to claim 1, wherein the inorganic filler comprises a metal-based filler.

14. The porous moisture-sensitive member according to claim 1, wherein the porous moisture-sensitive member comprises a surfactant, and an amount of the surfactant is 0.1% by weight to 25% by weight with respect to the porous moisture-sensitive member.

15. A humidity sensor comprising:
a first electrode;
a second electrode; and
the porous moisture-sensitive member according to claim 1 between the first electrode and the second electrode.

16. The humidity sensor according to claim 15, further comprising a base plate, wherein the first electrode is on the base plate.

17. A respiration sensing system that senses respiration of a subject, the respiration sensing system comprising:
the humidity sensor according to claim 15 is disposed within a sphere region defined by a sphere that has a center as an exhalation source of the subject and has a radius of 150 cm or less.

18. The respiration sensing system according to claim 17, wherein the humidity sensor is disposed within a circular cone A region;
the circular cone A has an apex as the exhalation source and a rotation axis as a straight line extending from the exhalation source in a front direction of the subject,
a cone angle defined by a vertex angle of an isosceles triangle formed when the circular cone A is cut along a plane passing through the rotation axis is 40° or less,
a cone height defined by a straight line length from a base center to the apex is 100 cm or less, and
the exhalation source is a mouth of the subject.

19. The respiration sensing system according to claim 17, wherein the humidity sensor is disposed within a circular cone B region;
the circular cone B has an apex as the exhalation source and a rotation axis as a straight line extending downward by 30° and rightward or leftward by 30° with respect to a back direction from the exhalation source,
a cone angle defined by a vertex angle of an isosceles triangle formed when the circular cone B is cut along a plane passing through the rotation axis is 60° or less,
a cone height defined by a straight line length from a base center to the apex is 30 cm or less, and
the exhalation source is a mouth or a nostril of the subject.

20. The respiration sensing system according to claim 17,
wherein the humidity sensor is disposed within a circular cone C region;
the circular cone C has an apex as the exhalation source and a rotation axis as a straight line extending from the exhalation source in a downward direction of the subject,
a cone angle defined by a vertex angle of an isosceles triangle formed when the circular cone C is cut along a plane passing through the rotation axis is 60° or less,
a cone height defined by a straight line length from a base center to the apex is 70 cm or less, and
the exhalation source is a nostril of the subject.

21. The respiration sensing system according to claim 17, wherein a sum of a response time and a recovery time of the humidity sensor is 5 seconds or less.

22. The respiration sensing system according to claim 17, further comprising a control unit that responds to a respiration sensing result, wherein the control unit controls an ambient environment of the subject by an air conditioning device based on the respiration sensing result.

23. The respiration sensing system according to claim 19, further comprising a control unit that responds to a respiration sensing result, wherein the control unit controls an automobile.

24. The respiration sensing system according to claim 19, further comprising a sensor that measures second vibration data including a second vital sign other than respiration, wherein the second vital sign is sensed by subtracting first vibration data based on a respiration sensing result from the second vibration data.

25. The respiration sensing system according to claim 19, wherein a linear distance between the humidity sensor and the exhalation source is 5 cm or more.
